(19) United States
(12) Patent Application Publication
(10) Pub. No.: US 2026/0232340 A1
(43) Pub. Date: Aug. 13, 2026

(54) TISSUE PERFORATION SYSTEMS

(71) Applicant: Edwards Lifesciences Corporation, Irvine, CA (US)

(72) Inventors: Ofir Witzman, Harish (IL); Eitan Atias, Tel Aviv (IL); Peleg Harel, Haifa (IL)

(73) Assignee: Edwards Lifesciences Corporation, Irvine, CA (US)

(21) Appl. No.: 19/637,909

(22) Filed: Apr. 2, 2026

Related U.S. Application Data
(63) Continuation of application No. PCT/US2024/050558, filed on Oct. 9, 2024.
(60) Provisional application No. 63/589,104, filed on Oct. 10, 2023.

(51) Int. Cl. A61B 17/32; A61B 17/00

(52) U.S. Cl. CPC A61B 17/320016; A61B 2017/00247; A61B 2017/00331; A61B 2017/32006

(57) ABSTRACT

The present disclosure relates to tissue perforation systems that can be used to form an opening in a target tissue, such as a host leaflet within which a guest prosthetic valve can be expanded. In an example, a tissue perforation system comprises a needle having a needle wall defining a needle lumen around a central axis of the needle, and at least one extendable protrusion that . continuously from a protrusion base at the needle wall to a protrusion tip. The radial distance between the protrusion tip and the central axis in a bent state of the needle is greater than a radial distance between the protrusion tip and the central axis in an unbent state of the needle, at least when the needle is not constrained in the bent state by an outer enclosure.

### CROSS-REFERENCE TO RELATED APPLICATIONS

This application is a continuation of International Application No. PCT/US2024/050558, filed October 9, 2024, which claims the benefit of U.S. Provisional Application No. 63/589,104, filed October 10, 2023, which is incorporated by reference herein.

### FIELD

The present disclosure relates to devices and systems configured to form an opening in the target tissue, and to methods and systems for puncturing through a target tissue that can be a leaflet of an existing valvular structure, in a manner that can modify existing valvular structures (for example, leaflets of a native heart valve or previously-implanted prosthetic valve) prior to implantation of a guest prosthetic valve.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches, such as transcatheter aortic valve replacement (TAVR), are used in various procedures to deliver prosthetic medical devices to locations inside the body that are not readily accessible by surgery or where access without surgery is desirable.

Transcatheter aortic valve replacement (TAVR) is one example of a minimally-invasive surgical procedure used to replace a native aortic valve. In one specific example of the procedure, an expandable prosthetic heart valve is mounted in a crimped state on the distal end of a delivery apparatus and advanced through the patient’s vasculature (for example, through a femoral artery and the aorta) to the heart. The prosthetic heart valve is positioned within the native valve and expanded to its functional size.

A variant of TAVR is valve-in-valve (ViV) TAVR, where a new prosthetic heart valve replaces a previously implanted prosthetic valve. In one specific example of the procedure, a new expandable prosthetic heart valve ("guest valve") is delivered to the heart in a crimped state, as described above for the "native" TAVR. The guest valve is positioned within the previously implanted prosthetic valve ("host valve") and then expanded to its functional size. The host valve in a ViV TAVR procedure can be a surgically implanted prosthetic valve or a transcatheter prosthetic valve. The term "host valve" is also used herein to refer to the native aortic valve in a native TAVR procedure.

### SUMMARY

One known technique for mitigating the risk of coronary ostial obstruction involves lacerating or severing a portion of one or more leaflets of the host valve (which can be an aortic bioprosthetic valve or a native aortic valve). Lacerating or severing a portion of the leaflet(s) reduces the risk of blocking the coronary ostia when the guest prosthetic valve is implanted and displaces the leaflets of the host valve toward the inner wall of the aortic root. However, method that rely on lacerating existing leaflets, require high spatial precision and surgical skill. Moreover, once the leaflets have been lacerated, the existing heart valve may function poorly and increase the risk of aortic insufficiency, at least until a replacement prosthetic valve has been successfully implanted. If the existing leaflets have become calcified, there is a further risk that the lacerating will release particulate or other debris into the blood stream, which may make the patient susceptible to vascular occlusion or stroke.

According to some aspects of the disclosure, there is provided a tissue perforating system, comprising an outer shaft and a needle axially movable through and relative to the outer shaft.

In some examples, the needle comprises a needle distal end portion comprising a needle tip, and a needle wall defining a needle lumen around a central axis of the needle.

In some examples, the needle further comprises at least one extendable protrusion extending continuously from a protrusion base at the needle wall, along at least one protrusion free edge, to a protrusion tip.

In some examples, the needle is configured to transition between an unbent state and a bent state.

In some examples, when the needle is in the bent state, the protrusion tip radially protrudes from the needle wall.

In some examples, when the needle is in the bent state, the protrusion tip is separated from the needle wall by a first radial distance, and when the needle is in the bent state, the protrusion tip is separated from the needle wall by a second radial distance, wherein the second radial distance is greater than the first radial distance.

In some examples, when the needle is in the bent state, the protrusion tip is separated from the central axis of the needle by a first radial distance, and when the needle is in the bent state, the protrusion tip is separated from the central axis of the needle by a second radial distance, wherein the second radial distance is greater than the first radial distance.

In some examples, the tissue perforating system further comprises an outer enclosure configured to allow the needle tip to radially protrude from the needle wall when the at least one protrusion is not constrained by the outer enclosure.

In some examples, the needle is in the bent state, the at least one extendable protrusion extends at an angle with respect to the needle wall.

In some examples, the needle distal end portion comprises an angled surface extending between the needle tip and a heel radially opposite to the needle tip.

In some examples, the protrusion tip of at least one of the at least one extendable protrusion is aligned with the heel in the unbent state of the needle.

In some examples, the tissue perforating system further comprises a covering member defining a covering member lumen through which the needle extends.

In some examples, the covering member defines the outer enclosure.

In some examples, the tissue perforating system further comprises an expansion member configured to expand a pilot puncture formed in a target tissue by the needle.

In some examples, the tissue perforating system further comprises a balloon catheter defining a balloon catheter lumen, wherein the expansion member comprises a balloon mounted on the balloon catheter and in fluid communication with the balloon catheter lumen, the balloon configured to transition between deflated and inflated states thereof.

In some examples, the tissue perforating system further comprises a dilator attached to a dilator shaft extending proximally therefrom through the balloon catheter lumen, wherein the needle extends through a dilator lumen defined by the dilator and the dilator shaft.

In some examples, at least part of the outer enclosure is defined by the dilator.

In some examples, at least part of the outer enclosure is defined by the dilator shaft.

In some examples, the needle is biased to the bent state in a free state thereof.

According to some aspects of the disclosure, there is provided a method comprising advancing a tissue perforating system comprising a needle, over a guidewire, to a host valvular structure.

In some examples, the method comprises transitioning the needle to a bent state thereof, such that at least one extendable protrusion of the needle extends radially outwards relative to a central axis defined by the needle.

In some examples, the method further comprises forming, with a needle tip of the needle, a pilot puncture within a host leaflet of the host valvular structure.

In some examples, the method comprises distally advancing the needle to cut through the host leaflet with the at least one extendable protrusion, thereby enlarging the pilot puncture.

In some examples, the transitioning the needle to the bent state comprises uncovering the needle.

In some examples, the at least one extendable protrusion extends continuously from a protrusion base at a needle wall of the needle, to a protrusion tip, defining at least one protrusion free edge between the protrusion base and the protrusion tip.

In some examples, the protrusion tip is distal to the protrusion base.

In some examples, the transitioning the needle to the bent state comprises distancing the protrusion tip to a distance that is greater than an outer radius of the needle, relative to the central axis.

In some examples, the at least one extendable protrusion comprises a plurality of extendable protrusions, and the distally advancing the needle to cut through the host leaflet with the at least one extendable protrusion comprises distally advancing the needle to cut through the host leaflet with the at least some of the plurality of extendable protrusions.

In some examples, the transitioning the needle to the bent state comprises pressing the needle tip against the host leaflet at a force that facilitates bending of the needle, yet is lower than a force required to penetrate the host leaflet by the needle tip.

In some examples, the forming the pilot puncture comprises applying a distally oriented force on the needle, sufficient to facilitate penetration of the needle tip through the host leaflet.

In some examples, the needle is configured to bias towards the bent state in a free state of the needle.

In some examples, the advancing the tissue perforating system to the host valvular structure comprises retaining the at least one extendable protrusion inside an outer enclosure of the tissue perforating system.

In some examples, the transitioning the needle to the bent state comprises exposing a portion of the needle that comprises the at least one extendable protrusion out of the outer enclosure, thereby allowing the exposed portion of the needle to assume the bent state.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

### IN THE FIGURES

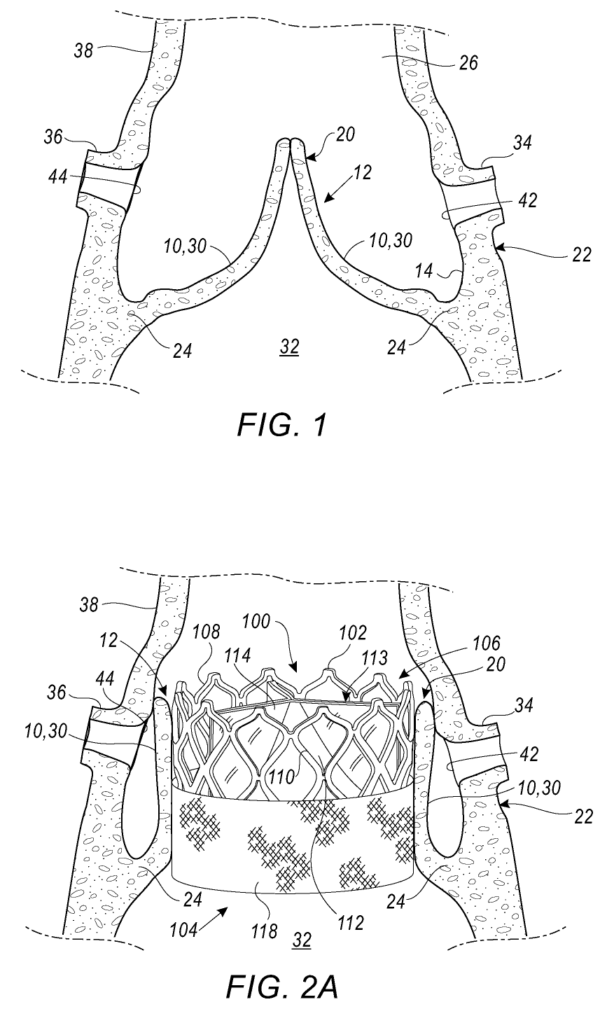

FIG. 1 is a cross-sectional view of a native aortic valve.

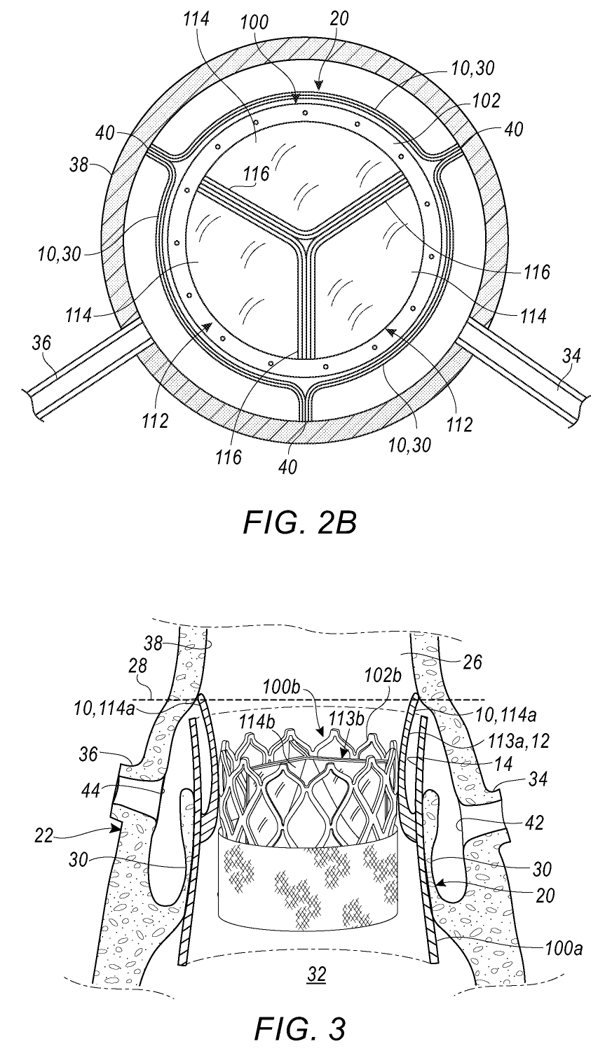

FIG. 2A shows a cross-sectional view of a prosthetic heart valve implanted in the native aortic valve of FIG. 1, according to an example.

FIG. 2B shows the implanted prosthetic heart valve of FIG. 1A as viewed from the ascending aorta, according to an example.

FIG. 3 shows a valve-in-valve implantation within the native aortic valve of FIG. 1, according to an example.

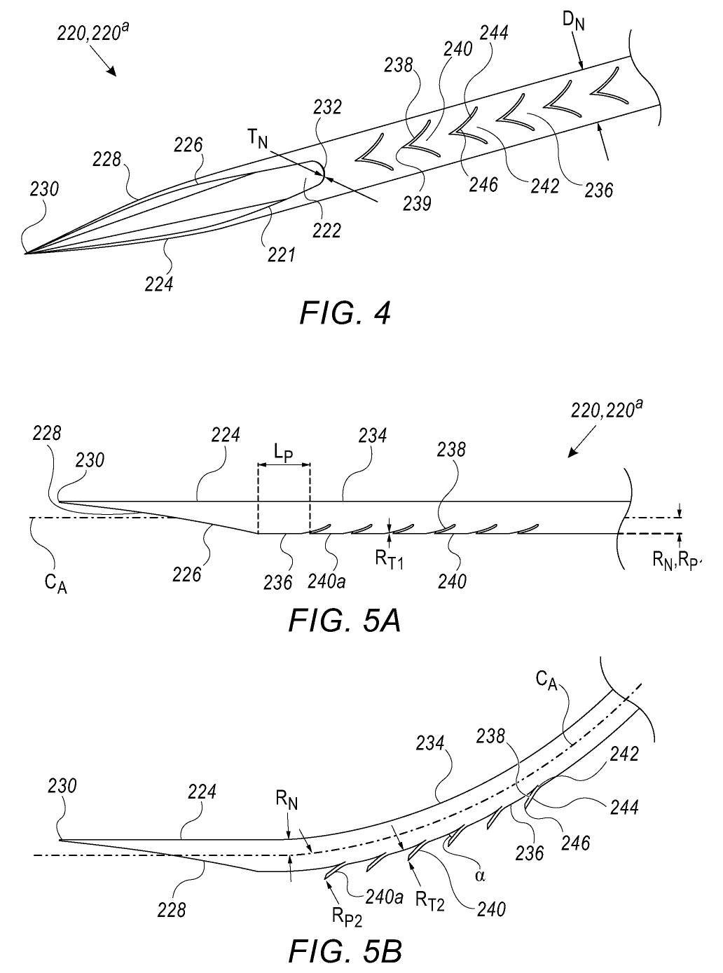

FIG. 4 is a perspective view of an exemplary hollow needle.

FIG. 5A is a side view of the needle of FIG. 4 in an unbent state.

FIG. 5B is a side view of the needle of FIG. 5A in a bent state.

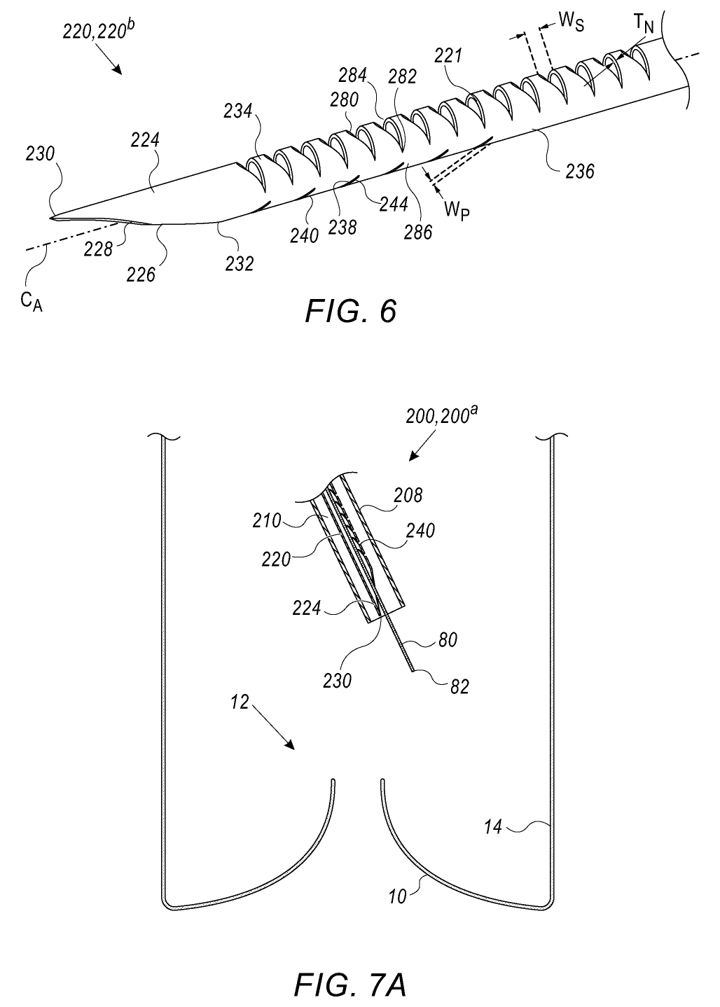

FIG. 6 is a perspective view of an exemplary needle that includes a plurality of successive discrete circumferential slots.

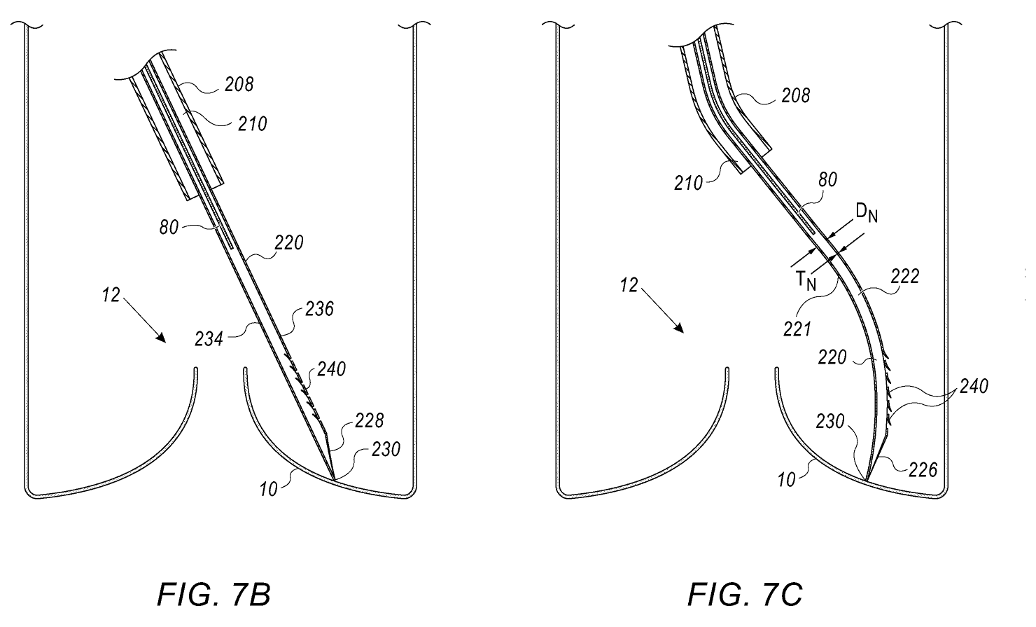

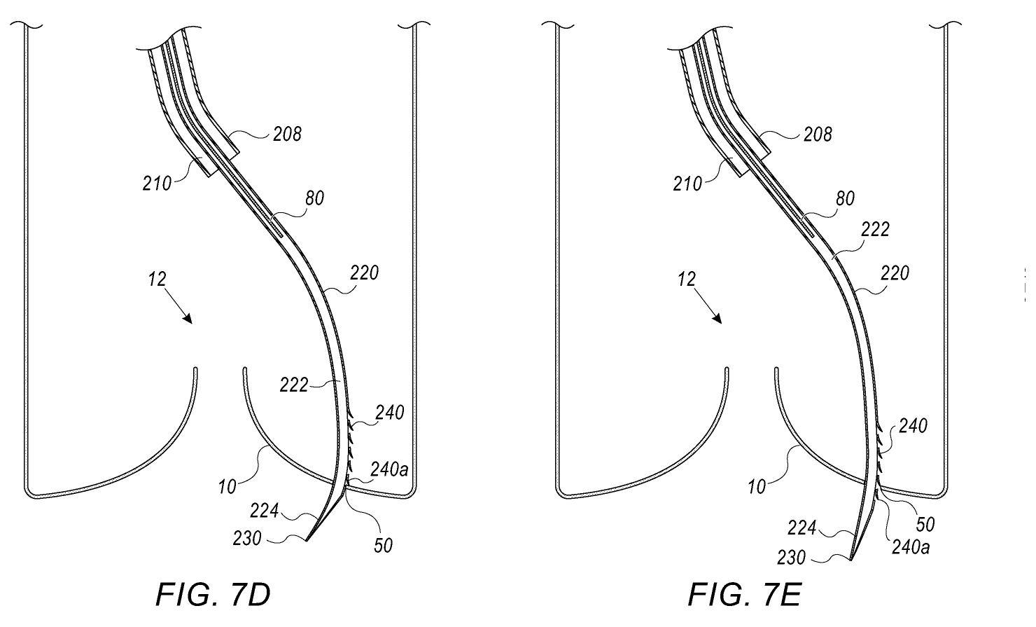

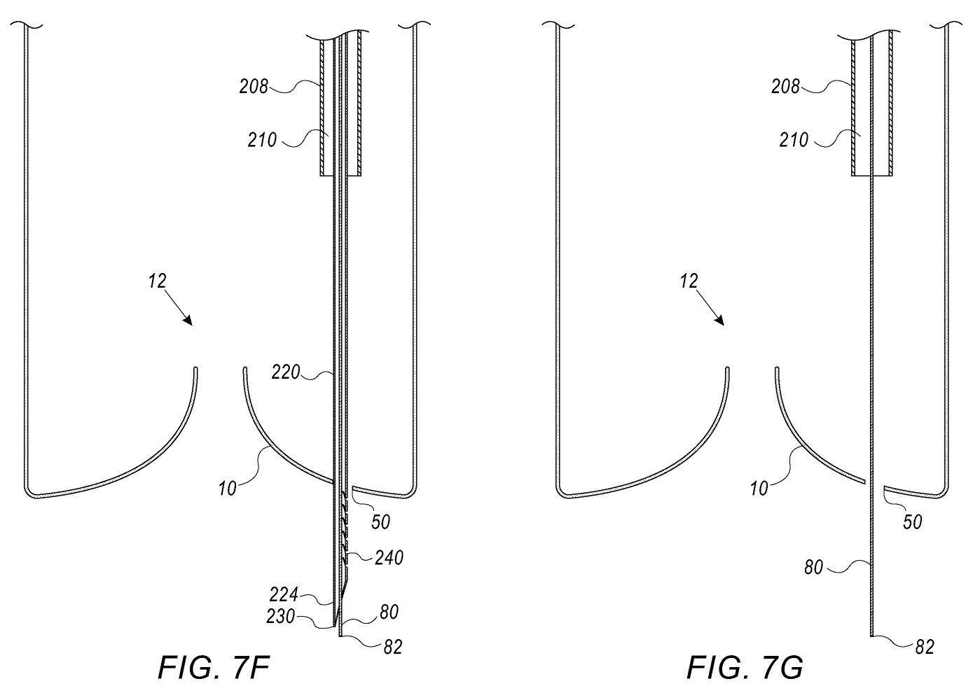

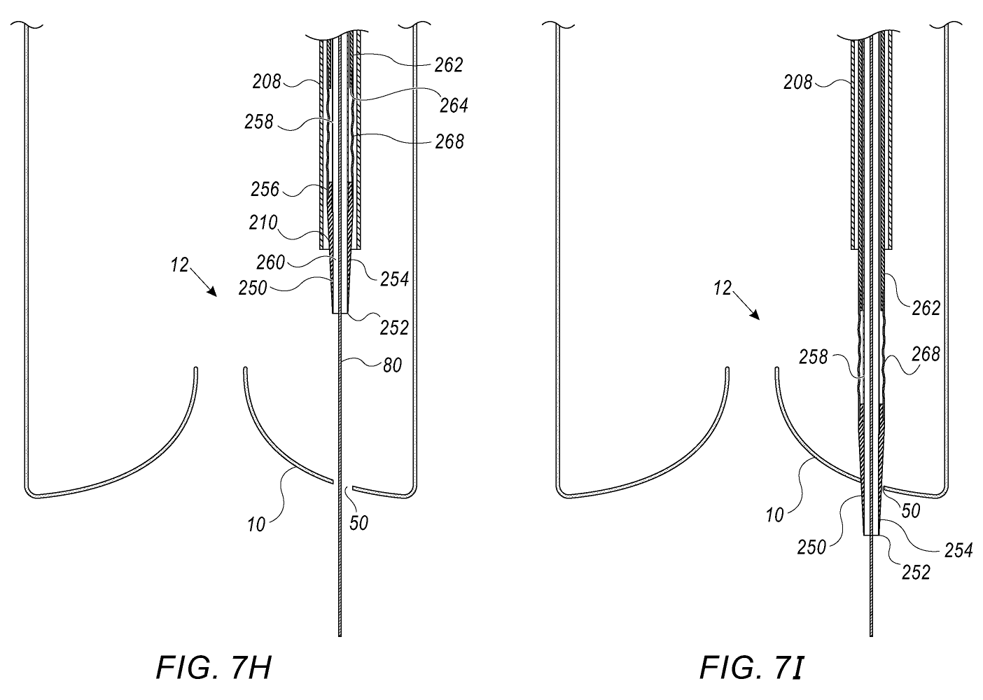

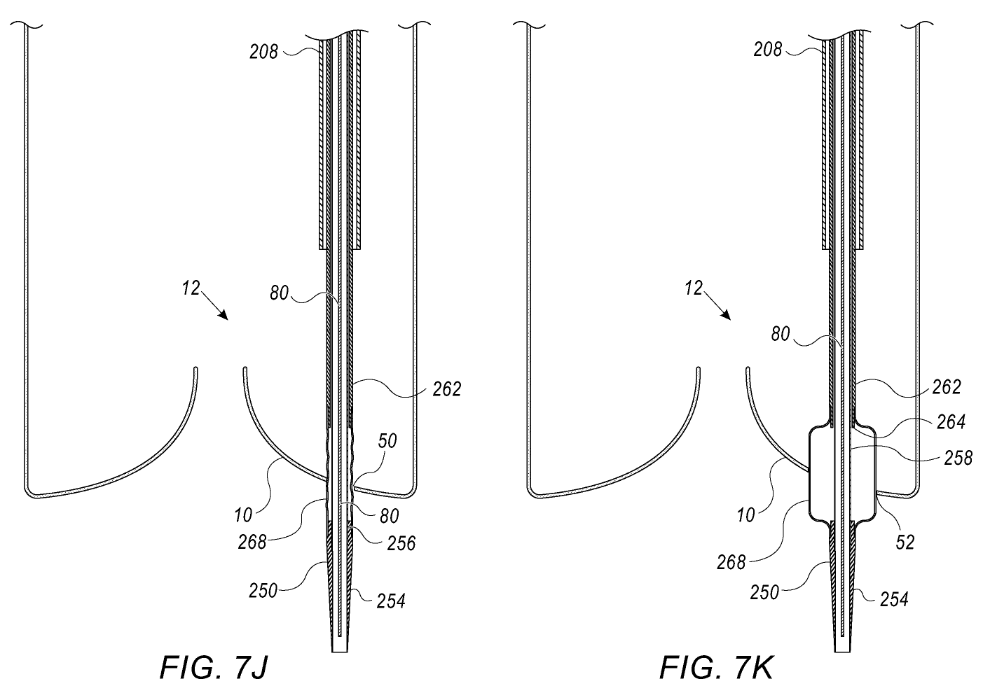

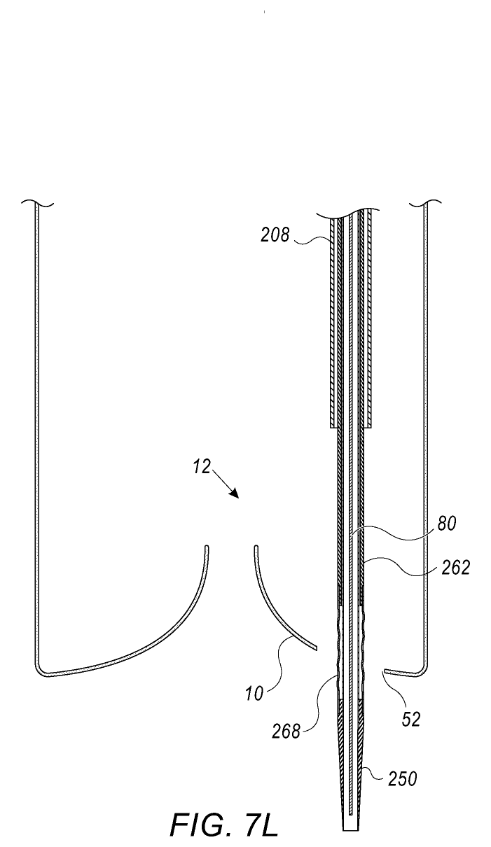

FIGS. 7A-7L illustrate steps in a method for utilizing an exemplary tissue perforating system that includes the needle of FIGS. 4-5B, for forming an opening within a host leaflet.

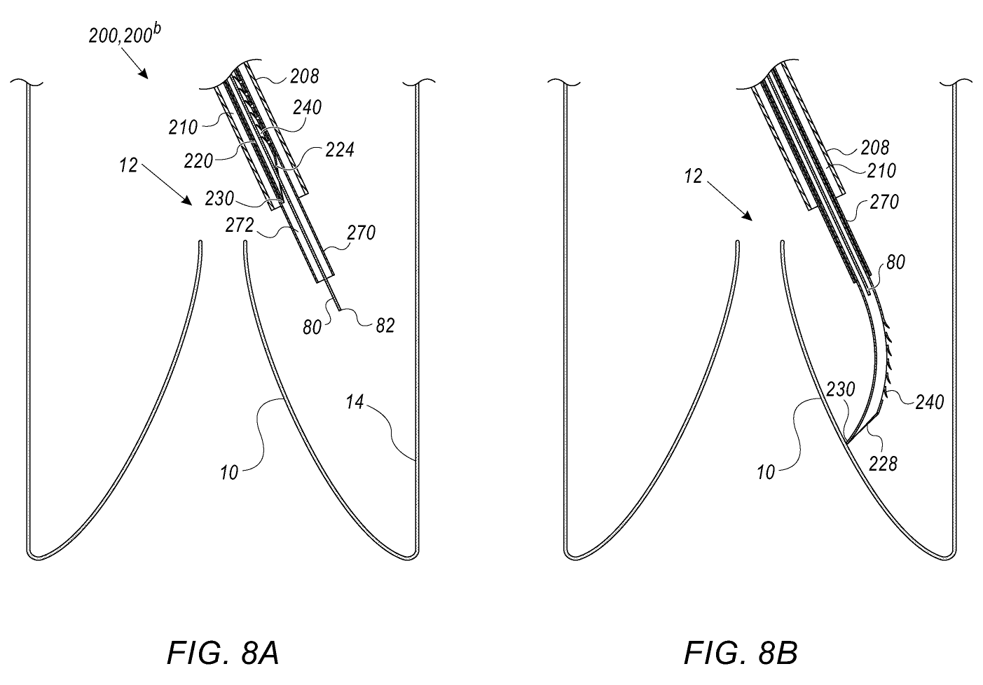

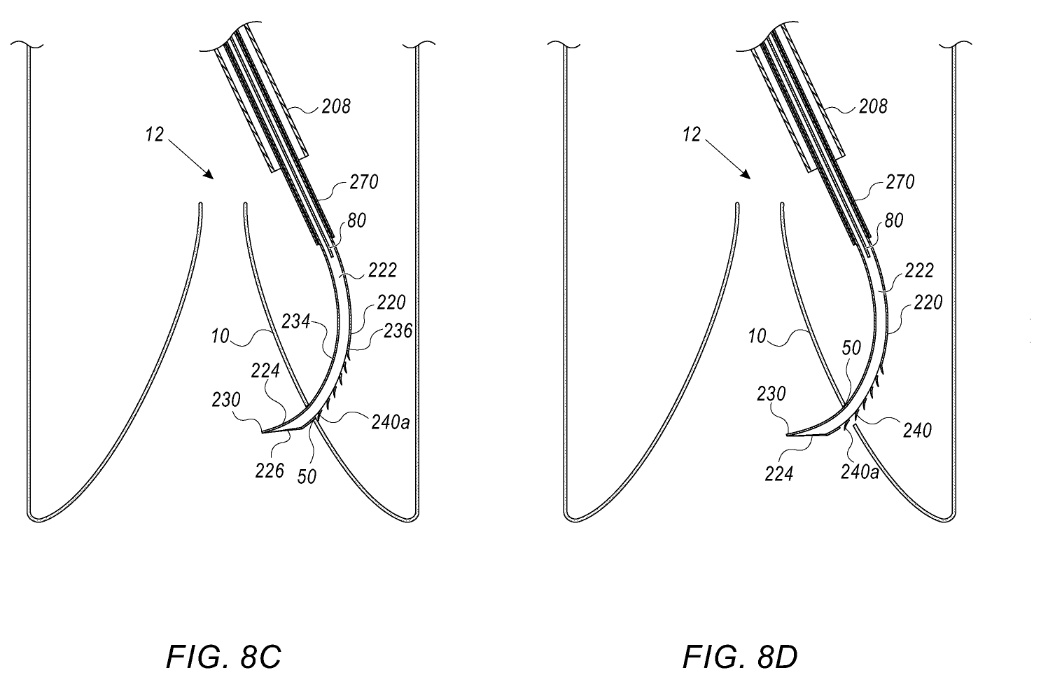

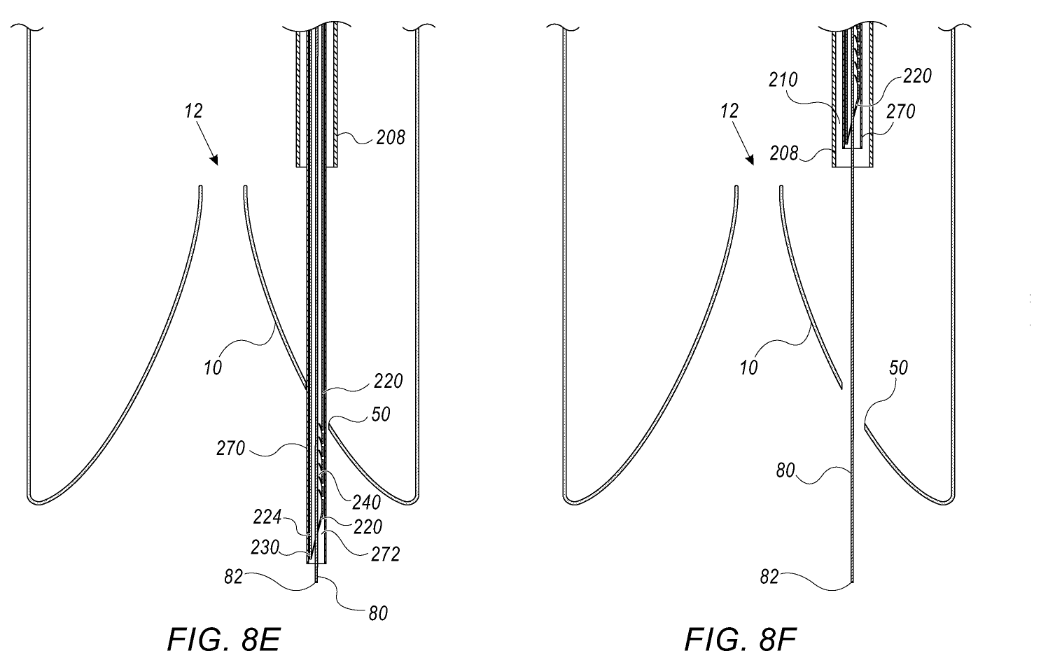

FIGS. 8A-8F illustrate steps in a method for utilizing an exemplary tissue perforating system that includes a covering member disposed around the needle, for forming an opening within a host leaflet.

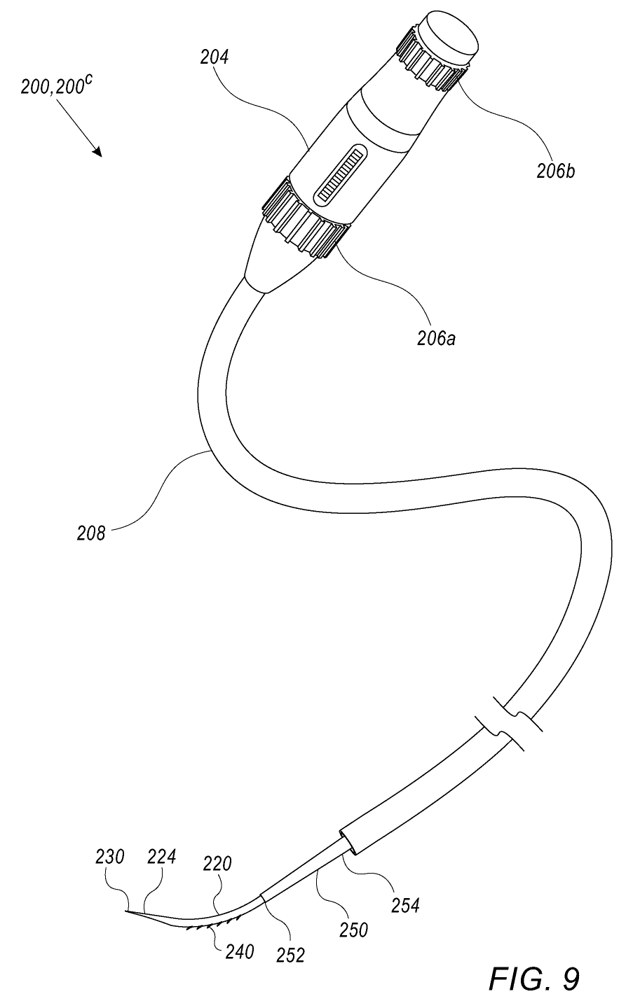

FIG. 9 illustrates an exemplary tissue perforation system.

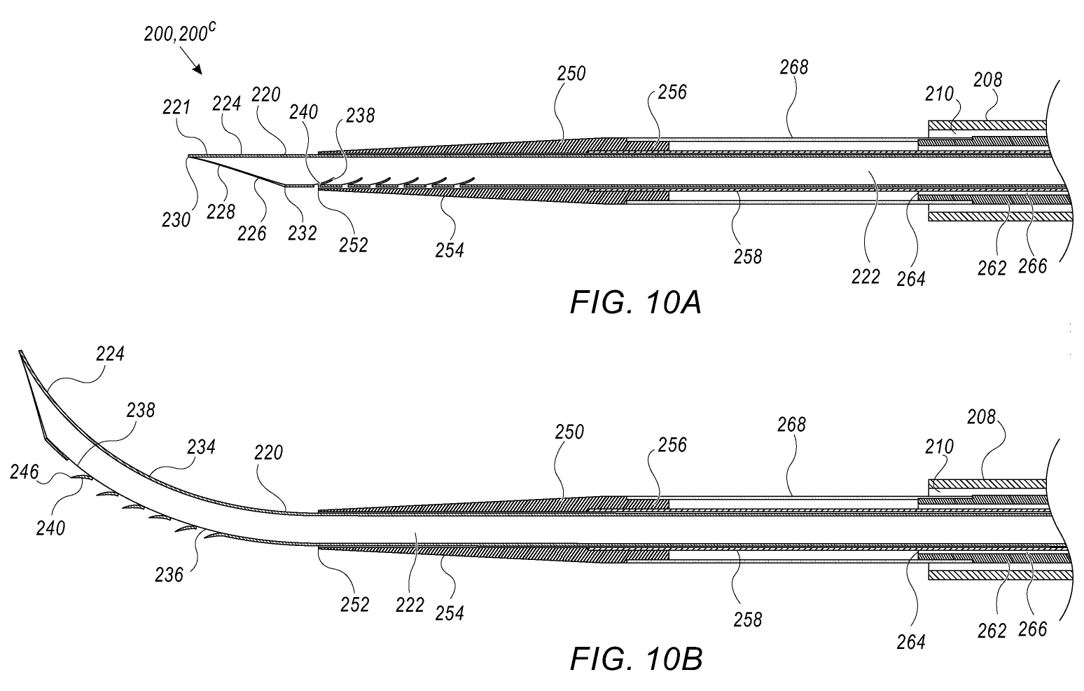

FIG. 10A is a side view of a distal portion of an exemplary tissue perforation system comprising a needle extending through a dilator lumen, in an unbent state of the needle.

FIG. 10B is a side view of a distal portion of the system of FIG. 10A, in a bent state of the needle.

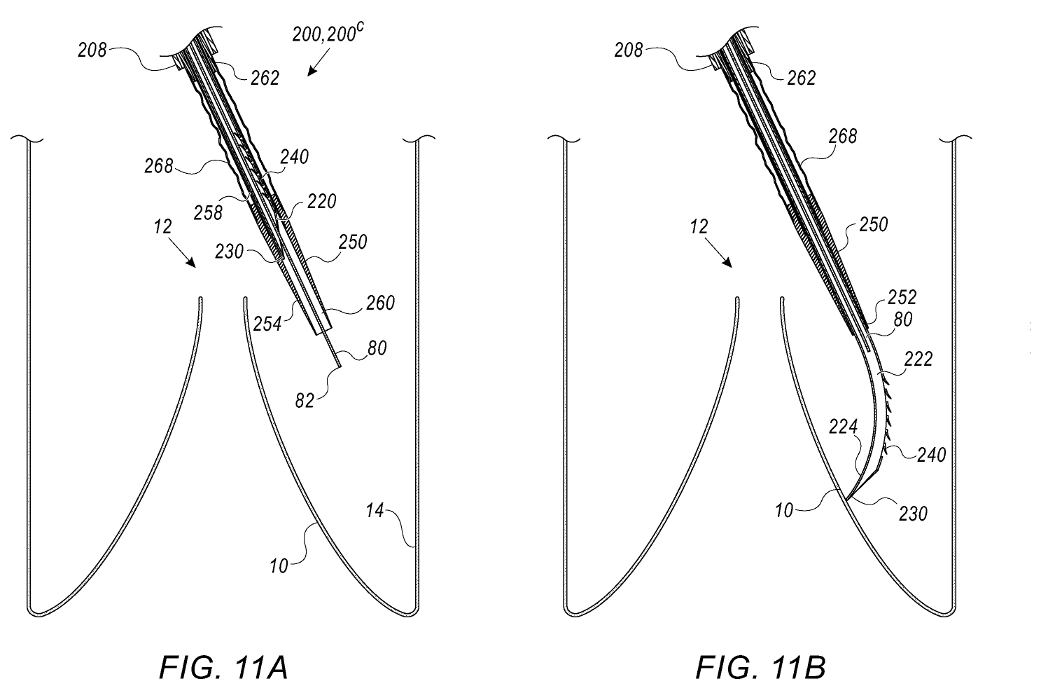

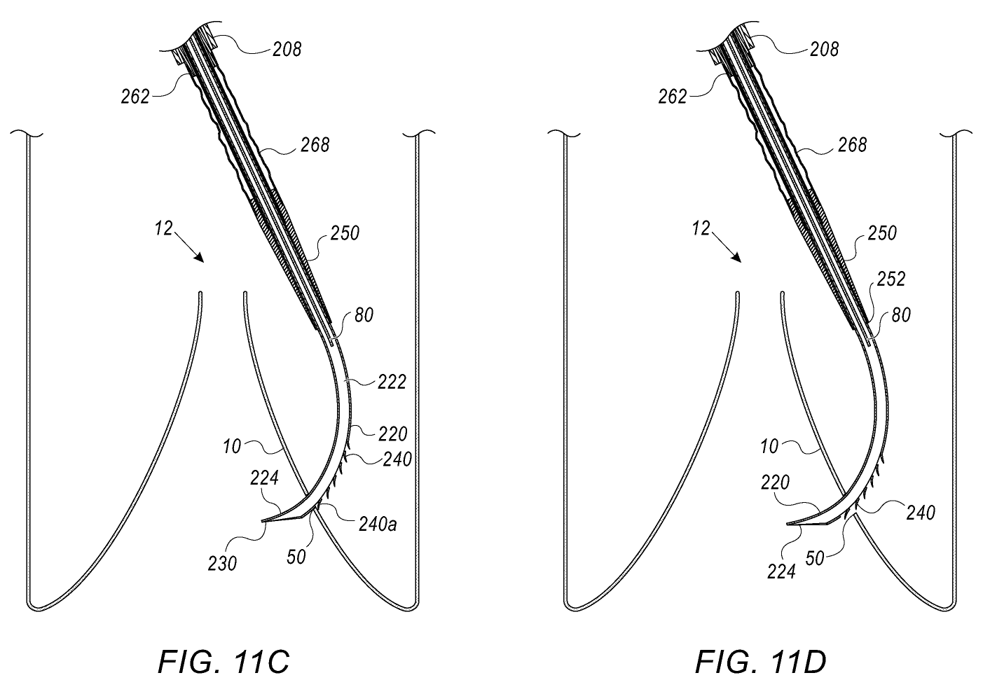

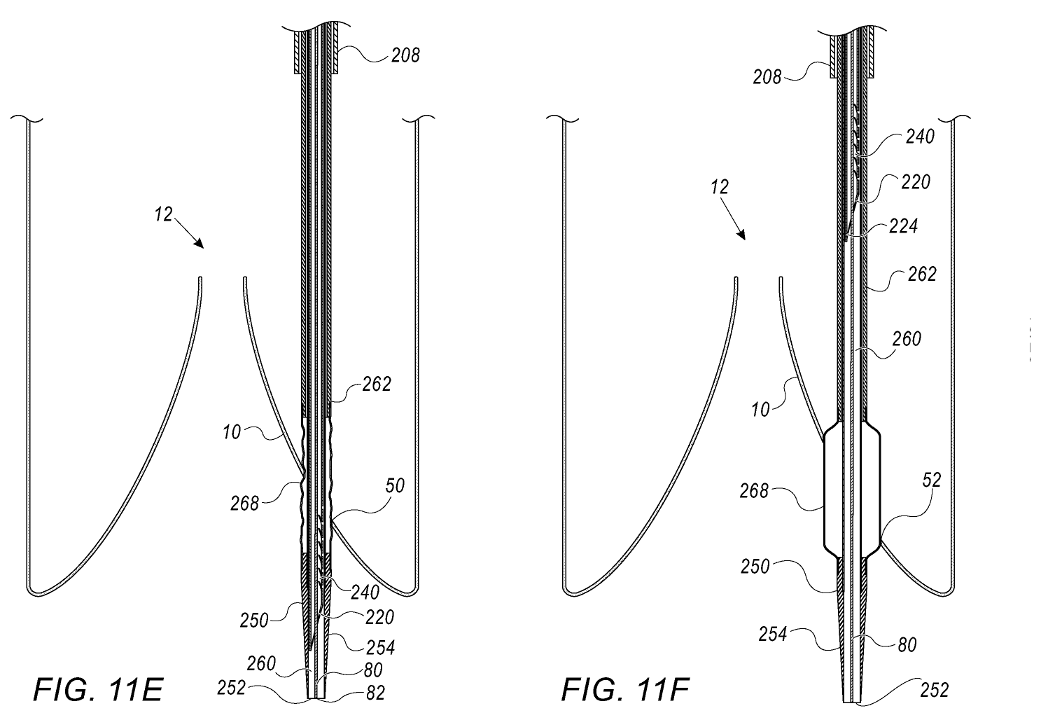

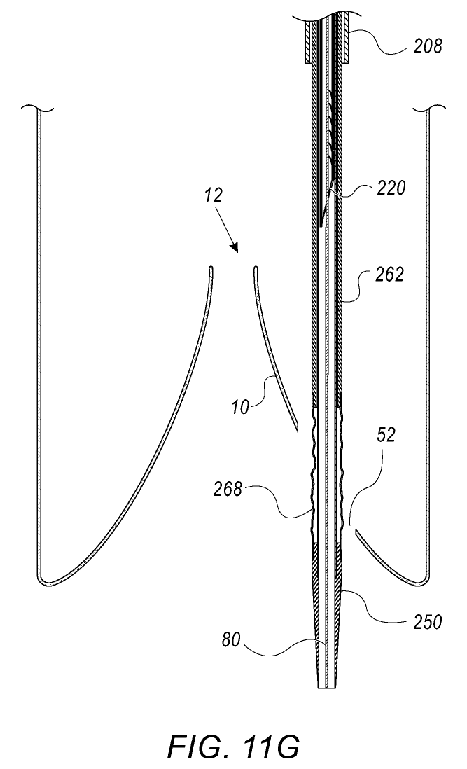

FIGS. 11A-11G illustrate steps in a method for utilizing the system of FIGS. 11A-11B, for forming an opening within a host leaflet.

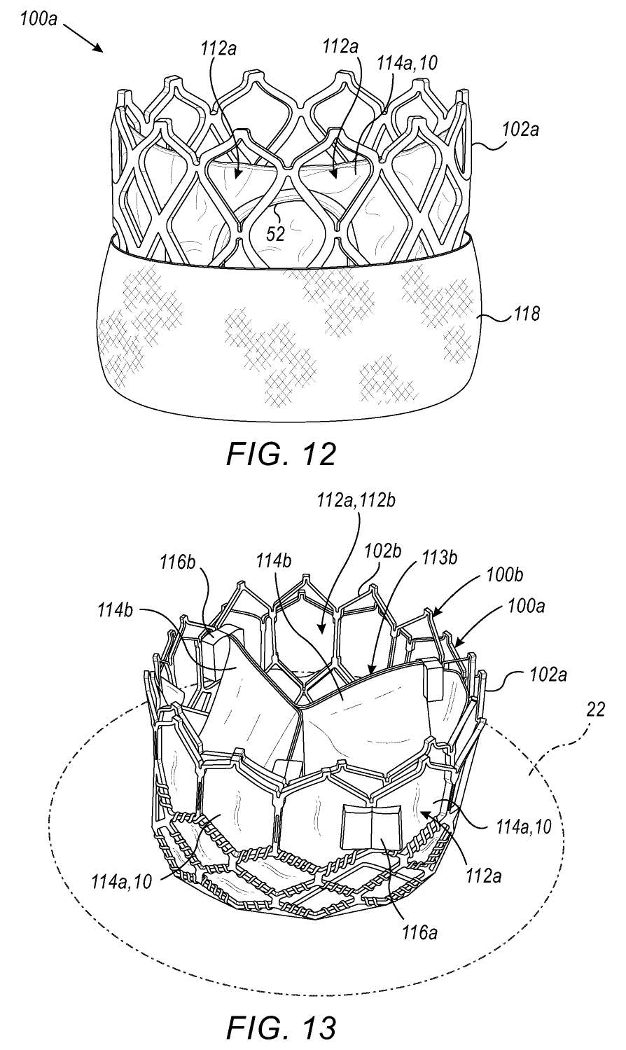

FIG. 12 is a perspective view of a host prosthetic valve subsequent to forming a leaflet opening thereof.

FIG. 13 is a perspective view of a guest prosthetic valve expanded within a leaflet opening of a host prosthetic valve.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like "provide" or "achieve" to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms "a", "an", and "the" include the plural forms unless the context clearly dictates otherwise. Additionally, the terms "have" or "includes" means "comprises". Further, the terms "coupled", "connected", and "attached", as used herein, are interchangeable and generally mean physically, mechanically, chemically, magnetically, and/or electrically coupled or linked and does not exclude the presence of intermediate elements between the coupled or associated items absent specific contrary language. As used herein, "and/or" means "and" or "or", as well as "and" and "or".

Directions and other relative references may be used to facilitate discussion of the drawings and principles herein, but are not intended to be limiting. For example, certain terms may be used such as "inner", "outer", "upper", "lower", "inside", "outside", "top", "bottom", "interior", "exterior", "left", right", and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an "upper" part can become a "lower" part simply by turning the object over. Nevertheless, it is still the same part and the object remains the same.

The term "plurality" or "plural" when used together with an element means two or more of the element. Directions and other relative references (for example, inner and outer, upper and lower, above and below, left and right, and proximal and distal) may be used to facilitate discussion of the drawings and principles herein but are not intended to be limiting.

The terms "proximal" and "distal" are defined relative to the use position of a delivery apparatus. In general, the end of the delivery apparatus closest to the user of the apparatus is the proximal end, and the end of the delivery apparatus farthest from the user (for example, the end that is inserted into a patient’s body) is the distal end. The term "proximal" when used with two spatially separated positions or parts of an object can be understood to mean closer to or oriented towards the proximal end of the delivery apparatus. The term "distal" when used with two spatially separated positions or parts of an object can be understood to mean closer to or oriented towards the distal end of the delivery apparatus. The terms "longitudinal" and "axial" are interchangeable, and refer to an axis extending in the proximal and distal directions, unless otherwise expressly defined.

The terms "axial direction", "radial direction", and "circumferential direction" have been used herein to describe the arrangement and assembly of components relative to the geometry of the frame of the prosthetic valve, or the geometry of an inflatable balloon that can be used to expand a prosthetic valve. Such terms have been used for convenient description, but the disclosed examples are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms "integrally formed" and "unitary" refer to a construction that does not include any welds, fasteners, or other means for securing separately formed pieces of material to each other.

As used herein, operations that occur "simultaneously" or "concurrently" occur generally at the same time as one another, although delays in the occurrence of operation relative to the other due to, for example, spacing between components, are expressly within the scope of the above terms, absent specific contrary language.

As used herein, terms such as "first", "second", and the like are intended to serve as respective labels of distinct components, steps, etc. and are not intended to connote or imply a specific sequence or priority. For example, unless otherwise stated, a step of performing a second action and/or of forming a second component may be performed prior to a step of performing a first action and/or of forming a first component.

As used herein, the term "substantially" means the listed value and/or property and any value and/or property that is at least 75% of the listed value and/or property. Equivalently, the term "substantially" means the listed value and/or property and any value and/or property that differs from the listed value and/or property by at most 25%. For example, "at least substantially parallel" refers to directions that are fully parallel, and to directions that diverge by up to 22.5 degrees.

In the present disclosure, a reference numeral that includes an alphabetic label (for example, "a", "b", "c", etc.) is to be understood as labeling a particular example of the structure or component corresponding to the reference numeral. Accordingly, it is to be understood that components sharing like names and/or like reference numerals (for example, with different alphabetic labels or without alphabetic labels) may share any properties and/or characteristics as disclosed herein even when certain such components are not specifically described and/or addressed herein.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

Described herein are devices and methods for implanting prosthetic valves and modifying leaflets of an existing valvular structure in a patient’s heart. Prior to or during implantation of the prosthetic heart valve within the existing valvular structure, each device, such as a delivery apparatus that can optionally carry a prosthetic valve, can be provided in the ascending aorta of a patient and can be used to pierce, lacerate, slice, tear, cut or otherwise modify a leaflet or commissure of the existing valvular structure. In some examples, the existing valvular structure can be a native aortic valve (for example, normal or abnormal, such as bicuspid aortic valve (BAV)) or a prosthetic valve previously implanted in the native aortic valve. The modification can avoid, or at least reduce the likelihood of, issues that leaflets of the existing valvular structure might otherwise cause once the prosthetic heart valve has been fully installed, for example, obstruction of blood flow to the coronary arteries, improper mounting due to a non-circular valve cross-section, and/or restricted access to the coronary arteries if subsequent intervention is required. While described with respect to aortic valve, it should be understood that the disclosed examples can be adapted to deliver devices that can modify existing valvular structure, and in some implementations, implant prosthetic devices, to and/or in any of the native annuluses of the heart (for example, the aortic, pulmonary, mitral, and tricuspid annuluses), and can be used with any of various delivery approaches (for example, retrograde, antegrade, transseptal, transventricular, transatrial, etc.).

FIG. 1 illustrates an anatomy of the aortic root 22, which is positioned between the left ventricle 32 and the ascending aorta 26. The aortic root 22 includes a native aortic valve 20 having a native valvular structure 29 comprising a plurality of native leaflets 30. Normally, the native aortic valve 20 has three leaflets (only two leaflets are visible in the simplified illustration of FIG. 1), but aortic valves with fewer than three leaflets are possible. The leaflets 30 are supported at native commissures by the aortic annulus 24, which is a ring of fibrous tissue at the transition point between the left ventricle 32 and the aortic root 22. The leaflets 30 can cycle between open and closed positions (the closed position is shown in FIG. 1) to regulate flow of blood from the left ventricle 32 to the ascending aorta 26. Branching off the aortic root 22 are the coronary arteries 34, 36. The coronary artery ostia 42, 44 are the openings that connect the aortic root 22 to the coronary arteries 34, 36.

FIGS. 2A-2B show an exemplary prosthetic valve 100 that can be implanted in a native heart valve, such as the native aortic valve 20 of FIG. 1. The term "prosthetic valve", as used herein, refers to any type of a prosthetic valve deliverable to a patient's target site over a catheter, which is radially expandable and compressible between a radially compressed, or crimped, state, and a radially expanded state. Thus, the prosthetic valve can be crimped on or retained by an implant delivery apparatus (not shown) in the radially compressed state during delivery, and then expanded to the radially expanded state once the prosthetic valve reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state. A prosthetic valve of the current disclosure (for example, prosthetic valve 100) may include any prosthetic valve configured to be mounted within the native aortic valve, the native mitral valve, the native pulmonary valve, and the native tricuspid valve.

It is understood that the prosthetic valves disclosed herein may be used with a variety of implant delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus (not shown). Self-expandable valves include a frame that is shape-set to automatically expand as soon an outer retaining shaft or capsule (not shown) is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Patent No. 10,603,165, International Application No. PCT/US2021/052745 and U.S. Provisional Application Nos. 63/085,947 and 63/209904, each of which is incorporated herein by reference in its entirety), releasably coupled to respective actuation assemblies of a delivery apparatus, controlled via a handle (not shown) for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

FIGS. 2A-2B show an example of a prosthetic valve 100, which can be a balloon expandable valve or any other type of valve, illustrated in an expanded state. The prosthetic valve 100 can comprise an outflow end 106 and an inflow end 104. In some instances, the outflow end 106 is the proximal end of the prosthetic valve 100, and the inflow end 104 is the distal end of the prosthetic valve 100. Alternatively, depending for example on the delivery approach of the valve, the outflow end can be the distal end of the prosthetic valve, and the inflow end can be the proximal end of the prosthetic valve.

The term "outflow", as used herein, refers to a region of the prosthetic valve through which the blood flows through and out of the prosthetic valve 100.

The term "inflow", as used herein, refers to a region of the prosthetic valve through which the blood flows into the prosthetic valve 100.

In the context of the present application, the terms "lower" and "upper" are used interchangeably with the terms "inflow" and "outflow", respectively. Thus, for example, the lower end of the prosthetic valve is its inflow end and the upper end of the prosthetic valve is its outflow end.

In the context of the present application, the terms "lower" and "upper" are used interchangeably with the terms "distal to" and "proximal to", respectively. Thus, for example, a lowermost component can refer to a distal-most component, and an uppermost component can similarly refer to a proximal-most component.

The terms "longitudinal" and "axial", as used herein, refer to an axis extending in the proximal and distal directions, unless otherwise expressly defined.

The prosthetic valve 100 comprises an annular frame 102 movable between a radially compressed configuration and a radially expanded configuration, and a valvular structure 113 that comprises prosthetic valve leaflets 114 mounted within the frame 102. The frame 102 can be made of various suitable materials, including plastically-deformable materials such as, but not limited to, stainless steel, a nickel based alloy (for example, a cobalt-chromium or a nickel-cobalt-chromium alloy such as MP35N alloy), polymers, or combinations thereof. When constructed of a plastically-deformable materials, the frame 102 can be crimped to a radially compressed state on a balloon catheter, and then expanded inside a patient by an inflatable balloon or equivalent expansion mechanism. Alternatively or additionally, the frame 102 can be made of shape-memory materials such as, but not limited to, nickel titanium alloy (for example, Nitinol). When constructed of a shape-memory material, the frame 102 can be crimped to a radially compressed state and restrained in the compressed state by insertion into a shaft or equivalent mechanism of a delivery apparatus.

In the example illustrated in FIGS. 2A-2B, the frame 102 is an annular, stent-like structure comprising a plurality of intersecting struts 108. In this application, the term "strut" encompasses axial struts, angled struts, laterally extendable struts, commissure windows, commissure support struts, support posts, and any similar structures described by U.S. Pat. Nos. 7,993,394 and 9,393,110, which are incorporated herein by reference. A strut 108 may be any elongated member or portion of the frame 102. The frame 102 can include a plurality of strut rungs that can collectively define one or more rows of cells 110. The frame 102 can have a cylindrical or substantially cylindrical shape having a constant diameter from the inflow end 104 to the outflow end 106 as shown, or the frame can vary in diameter along the height of the frame, as disclosed in US Pat. No. 9,155,619, which is incorporated herein by reference.

The struts 108 can include a plurality of angled struts and vertical or axial struts. At least some of the struts 108 can be pivotable or bendable relative to each other, so as to permit frame expansion or compression. For example, the frame 102 can be formed from a single piece of material, such as a metal tube, via various processes such as, but not limited to, laser cutting, electroforming, and/or physical vapor deposition, while retaining the ability to collapse/expand radially in the absence of hinges and like.

A valvular structure 113 of the prosthetic valve 100 can include a plurality of prosthetic valve leaflets 114 (for example, three leaflets), positioned at least partially within the frame 102, and configured to regulate flow of blood through the prosthetic valve 100 from the inflow end 104 to the outflow end 106. While three leaflets 114 arranged to collapse in a tricuspid arrangement, are shown in the example illustrated in FIGS. 2A-2B, it will be clear that a prosthetic valve 100 can include any other number of leaflets 114. Adjacent leaflets 114 can be arranged together to form prosthetic valve commissures 116 that are coupled (directly or indirectly) to respective portions of the frame 102, thereby securing at least a portion of the valvular structure 113 to the frame 102. The prosthetic valve leaflets 114 can be made from, in whole or part, biological material (for example, pericardium), bio-compatible synthetic materials, or other such materials. Further details regarding transcatheter prosthetic valves, including the manner in which leaflets 114 can be coupled to the frame 102 of the prosthetic valve 100, can be found, for example, in U.S. Patent Nos. 6,730,118, 7,393,360, 7,510,575, 7,993,394, 8,652,202, and 11,135,056, all of which are incorporated herein by reference in their entireties.

In some examples, the prosthetic valve 100 can comprise at least one skirt or sealing member. For example, the prosthetic valve 100 can include an inner skirt (not shown in FIG. 2A-2B), which can be secured to the inner surface of the frame 102. Such an inner skirt can be configured to function, for example, as a sealing member to prevent or decrease perivalvular leakage. An inner skirt can further function as an anchoring region for leaflets 114 to the frame 102, and/or function to protect the leaflets 114 against damage which may be caused by contact with the frame 102, for example during valve crimping or during working cycles of the prosthetic valve 100. An inner skirt can be disposed around and attached to the inner surface of frame 102, while the leaflets can be sutured to the inner skirt along a scalloped line (not shown). An inner skirt can be coupled to the frame 102 via sutures or another form of coupler.

The prosthetic valve 100 can comprise, in some examples, an outer skirt 118 mounted on the outer surface of frame 102 (as shown in FIGS. 2A-2B), configured to function, for example, as a sealing member retained between the frame 102 and the surrounding tissue of the native annulus against which the prosthetic valve is mounted, or against an inner side of a previously implanted valve in the case of ViV procedures (described further below), thereby reducing risk of paravalvular leakage (PVL) past the prosthetic valve 100. The outer skirt 118 can be coupled to the frame 102 via sutures or another form of coupler.

Any of the inner skirt and/or outer skirt can be made of various suitable biocompatible materials, such as, but not limited to, various synthetic materials (for example, PET) or natural tissue (for example pericardial tissue). In some cases, the inner skirt can be formed of a single sheet of material that extends continuously around the inner surface of frame 102. In some cases, the outer skirt 118 can be formed of a single sheet of material that extends continuously around the outer surface of frame 102.

The cells 110, defined by interconnected struts 108, define cell openings 112. While some of the cell openings 112 can be covered by the inner skirt and/or the outer skirt, at least a portion of the cell opening 112 can remain uncovered, such as cell openings 112 which are closer to the outflow end 106 of the prosthetic valve.

FIGS. 2A-2B illustrate a hypothetical coronary artery obstruction that could occur in some cases from implantation of a prosthetic valve 100 within the native aortic valve 20. In this example, the prosthetic valve 100 is the guest valve or new valve, and the native aortic valve 20 is the host valve or old valve.

During implantation of the prosthetic valve 100, the prosthetic valve 100 is positioned within a central region defined between the native leaflets 30, which are also the host leaflets 10 for the example illustrated in FIG. 2A-2B. The prosthetic valve 100 is then radially expanded against the host leaflets 10. As illustrated, the host leaflets 10 form a tube around the frame 102 of the prosthetic valve 100 after the prosthetic valve 100 is radially expanded to the working diameter. As further illustrated, expansion of the prosthetic valve 100 displaces the host leaflets 10 outwards towards the coronary ostia 42, 44 such that the host leaflets 10 contact a portion of the aortic root 22 surrounding the coronary ostia 42, 44, causing coronary artery obstruction.

For an existing implanted prosthetic valve, the valvular structure may naturally degrade over time thereby requiring repair or replacement in order to maintain adequate heart functions. In a Valve-in-Valve (ViV) procedure, a new prosthetic heart valve is mounted within the existing, degrading prosthetic heart valve in order to restore proper function. FIG. 3 illustrates an exemplary hypothetical coronary artery obstruction that could occur in some cases from implantation of a prosthetic valve 100b within a previously implanted prosthetic valve 100a (for example, after a ViV procedure). In this example, the prosthetic valve 100b is the guest valve or new valve, and the prosthetic valve 100a is the host valve or old valve. In this example, the prosthetic valve 100a was previously implanted within the orifice of the native aortic valve 20. Each of the prosthetic valves 100a, 100b can have the general structure of the prosthetic valve 100 described with reference to FIGS. 2A-2B, though in some examples, each of the prosthetic valves 100a, 100b can be a different type of prosthetic valve. For example, a balloon expandable guest valve 100b can be implanted inside a previously implanted mechanically expandable or self-expandable host valve 100a.

During implantation of the prosthetic valve 100b, the prosthetic valve 100b is positioned within a central region defined between the leaflets 114a of the prosthetic valve 100a, which now take the role of host leaflet 10. The prosthetic valve 100b is then radially expanded against the host leaflets 10 (i.e., against the prosthetic valve leaflets 114c). As illustrated, the radial expansion of the prosthetic valve 100a results in outward displacement of the host leaflets 10. As further illustrated, the host leaflets 10 are displaced such that the host leaflets 10 contact the aortic root 22 at positions superior to the coronary artery ostia 42, 44, causing coronary artery ostia obstruction. Alternatively, the guest prosthetic valve 100b can displace the host leaflets 114a outwardly against the frame 102a of the host valve 100a, thereby blocking the flow of blood through the frame 102a to the coronary ostia 42, 44.

In some patient anatomies (for example, when the outflow end 106 of the prosthetic valve 100 is at the STJ level 28 and the diameter of the prosthetic valve 100 is similar to the STJ diameter such that the frame 102 touches or is very close to the aortic wall 38 at the STJ level 28), the host leaflets 10 may compromise the ability for future access into the coronary arteries 34, 36 or perfusion through the frame 102 to the coronary arteries 34, 36 during the diastole phase of the cardiac cycle. Similar problems may occur in some patient anatomies either when a guest prosthetic valve 100b is percutaneously expanded within a previously implanted host prosthetic valve 100a, or when a prosthetic valve 100 is percutaneously expanded within a native valve, displacing the native leaflets 30 outward toward the coronary ostia 42, 44.

The risk illustrated in FIG. 3 may be higher when the host valve is a bioprosthetic valve without a frame or when the leaflets of the host valve are external to a frame. Risk of coronary artery ostia obstruction can increase in a cramped aortic root or when the coronary artery ostium sits low. In the examples illustrated in FIGS. 2A-3, the host leaflets 10 are shown obstructing both coronary artery ostia 42, 44. In some cases, only one host leaflet 10 may obstruct a respective coronary artery ostium. For example, the risk of obstructing the left coronary ostium 42 tends to be greater than obstructing the right coronary ostium 44 because the left coronary ostium 42 typically sits lower than the right coronary ostium 44.

The term "host valve" as used herein refers to a native heart valve in which a prosthetic valve is implanted or a previously implanted prosthetic valve in which a new prosthetic valve is implanted. Moreover, in any of the examples disclosed herein, when the host valve is a previously implanted prosthetic valve, the host valve can be a surgically implanted prosthetic heart valve (known as a "surgical valve") or a transcatheter heart valve. The term "guest valve", as used herein, refers to a prosthetic valve implanted in a host valve, which can be either a native heart valve or a previously implanted prosthetic valve. Similarly, the term "host leaflets 10", as used herein, refers to native leaflets 30 of a native valve in which a new guest prosthetic valve 100 is implanted, or to prosthetic valve leaflets 114a of a previously implanted host valve 100a in which a new guest prosthetic valve 100b is implanted.

When a guest prosthetic valve 100 is deployed inside a host valvular structure 12, it displaces the host leaflets 10 of the host valve radially outwards, towards and against a host interior surface 14, which can be the interior surface of the aortic wall 38 if the host valve is the native valve, or an interior surface of the frame 102a of a previously implanted prosthetic valve 100a serving as the host valve.

To avoid obstruction of blood flow to the coronary arteries 34, 36, the valvular structure 12 of the existing host valve (whether a native aortic valve or a previously implanted prosthetic valve) can be modified by components of a delivery apparatus prior to or during implantation of a new prosthetic valve within the existing valvular structure 12. In some examples, the host valvular structure 12 is modified by piercing, lacerating, tearing, slicing, and/or cutting one or more host leaflets 10 (for example, a free end of the host leaflet 10 or a commissure of adjacent host leaflets 10, which can be a native commissure 40 for a native aortic valve 20, or a prosthetic valve commissure 116 for a previously implanted host prosthetic valve 100) using the delivery apparatus. The modification thus disrupts the impermeable tubular structure that would otherwise be formed by the existing host leaflets 10, thereby allowing blood to flow to the coronary arteries 34, 36.

FIG. 4 is a perspective view of an exemplary hollow needle 220. FIGS. 5A and 5B are side views of the needle 220 of FIG. 4 in an unbent and a bent state, respectively. The needle 220 comprises a needle wall 221 that defines a needle lumen 222 having a tube central axis C_{A}. The needle 220 includes a needle distal end portion 224 configured to pierce a host leaflet 10 of a host valvular structure 12 to form a pilot puncture 50 in the host leaflet 10. A distal edge 226 of needle 220 can define an angled surface 228 extending between a sharp needle tip 230 configured to facilitate piercing the host leaflet 10 when the needle 220 is pressed against the leaflet, and a heel 232 of the angled surface 228, opposite to the needle tip 230. A tip side 234 of the needle 220 can be defined as the side aligned with the needle tip 230, and a heel side 236 can be similarly defined as the side of the needle 220 aligned with the heel 232, opposite to the tip side 234.

The needle wall 221 has a needle wall thickness T_{N}, and the needle 220 comprises at least one extendable protrusion 240 extending from a protrusion base 242, at which the protrusion 240 continuously extends from a portion of the needle wall 221 proximal thereto, to a protrusion tip 246 distal to the protrusion base 242, defining at least one free edge 244 extending between the base 242 and the tip 246. In some examples, the protrusion free edge 244 is formed as a sharp cutting edge. In some examples, the protrusion tip 246 is formed as a sharp penetrating tip. In the example illustrated in FIGS. 4-5B, the protrusion free edge 244 is shown to include two portions of the free edge 244 distally extending from circumferential opposite ends of the protrusion base 242, so as to converge at the protrusion tip 246, forming a V-shaped extendable protrusion 240. It is to be understood, however, that this shape of the extendable protrusion is shown by illustration and not limitation, and that other shapes of the extendable protrusion 240 are contemplated, such as a C-shaped extendable protrusion 240 (not shown) that can have a substantially semi-circular free edge 244 terminating at a curved protrusion tip 246, or any other suitable shape.

An extendable protrusion 240 can be formed by cutting the needle wall 221 (for example, by laser cutting) along the contour of the free edge 244, thus forming a relatively thin slit between a cut-out edge 238 of the needle wall 221 and the protrusion free edge 244, such that the extendable protrusion 240 can continuously extend from the needle wall 221 at the protrusion base 242, while the protrusion free edge 244 and protrusion tip 246 are unattached to the needle wall 221. Thus, it is to be understood that any reference throughout the current specification and claims, to a needle protrusion 240 protruding away from the needle wall 221 and/or having a protrusion tip 246 distanced from the needle wall 221, refers to the portion of the needle wall 221 that excludes protrusion(s) 240. The cut-out edges 238 of the needle wall 221 can converge at a cut-out tip 239, which is aligned with the protrusion tip 246, at least in the unbent state of the needle.

In some examples, a single extendable protrusion 240 can be provided. In some examples, a plurality of extendable protrusions 240 are serially provided, axially spaced from each other at equal or non-equal distances. Any reference to extendable protrusions 240 in the plural form throughout the specification, can similarly refer to a single extendable protrusion 240, unless stated otherwise. Similarly, while a plurality of extendable protrusions 240 are illustrated throughout FIGS. 4-11G, it is to be understood that any exemplary needle 220 disclosed and illustrated herein can include a single extendable protrusion 240, unless stated otherwise.

The extendable protrusions 240 can be formed along at least one side of the needle 220, such as the tip side 234 or the heel side 236, depending on the direction along which the needle 220 is configured to bend. As shown in FIG. 5A, when the needle is in an unbent state, the protrusions 240 can be flush with the rest of the needle wall 221, such that the protrusion free edges 244 and the protrusion tips 246 are at the same radial distance R_{N}, relative to the central axis C_{A}, as the rest of the needle wall 221, wherein R_{N} is the outer radius of the needle 220. As shown in FIG. 5B, when the needle is bent, the protrusion tip 246 radially protrudes from the needle wall 241. A radial distance R_{P} can be defined between the central axis C_{A} and the protrusion tip 246. A first radial distance R_{P1} is defined between the protrusion tip 246 and the central axis C_{A} in the unbent state of the needle. In some examples, the first radial distance R_{P1} can be equal to the needle's radius R_{N} in the unbent state of the needle. When the needle is bent, the unattached free edges 244 and tips 246 will cause the protrusions 240 to extend radially outwards, away from the central axis C_{A}, in a direction opposite to the direction of the needle's bending, such that the a second radial distance R_{P2} between the central axis C_{A} and the protrusion tip 246 of an extended protrusion is greater than the first radial distance R_{P1}.

As mentioned, the protrusion tip 246 is separated from the needle wall 261, such as from the corresponding cut-out tip 239, which is the closest part of the needle wall 241 to the protrusion tip 246. A protrusion tip 246 is separated from the protrusion wall 241 by a radial distance R_{T}, which can be a first radial distance R_{T1} in the unbent state of the needle, and can be a second radial distance R_{T2} in the bent state of the needle. As shown in FIG. 5B, when the needle is in the bent state, the protrusion tip 246 radially protrudes from the needle wall 241 such that the second radial distance R_{T2} is greater than the first radial distance R_{T1}. In some examples, such as when the protrusion 240 is flush with the rest of the needle wall 241 in the unbent state, as illustrated in FIG. 5A, the first radial distance R_{T1} can have a zero value. Nevertheless, in some examples (not shown), the needle tip 246 can be separated from the needle wall 241 by a small positive value of R_{T1}, and extends to a significantly greater second radial distance R_{T2} in the bent state. It is to be understood that a radial distance R_{T} between the protrusion tip 246 and the needle wall 241 refers to a radial distance measured between the protrusion tip 246 and the corresponding cut-out tip 239 of the needle wall 241.

As further shown in FIG. 5B, when the needle is in the bent state, the protrusion 240 can extend at an angle α with respect to the needle wall 241. The angle α can be defined between an imaginary line extending from the protrusion base 242 to the protrusion tip 246, and an imaginary line extending from the protrusion base 242 and the corresponding cut-out tip 239. When the protrusion 240 is flush with the rest of the needle wall 241 in the unbent state, the angle α can have a zero value, and when the protrusion 240 extends away from the needle wall 241 in the bent state, it can define a positive greater value of the α.

In general, the extendable protrusions 240 will be formed at the side of the needle 220 which is opposite to the direction of bending. In the examples illustrated in FIGS. 4-5B, the extendable protrusions 240 are formed at the heel side 236, such that the protrusion tips 246 are circumferentially aligned with the heel 232 of the needle 220. This position of the protrusion 240 will facilitate outwardly-directed extension thereof, when the bending direction of the needle 220 is as illustrated in FIG. 5B, such that the radius of curvature of the heel side 236 is greater than the radius of curvature of the tip side 234. In some examples, the extendable protrusions 240 can be formed at the tip side 234, such that the protrusion tips 246 are circumferentially aligned with the needle tip 230, which will facilitate outwardly-directed extension of the protrusions 240 when the needle 220 is bent in a direction opposite to that shown in FIG. 5B, for example such that the curvature of the tip side 234 is greater than the curvature of the heel side 236 (example not illustrated). In some examples, extendable protrusion 240 can be formed both along the heel side 236 and the tip side 234, which can be of benefit if the needle is configured to selectively bend in both directions, such that the appropriate extendable protrusions 240 can extend outwardly in response to the needle 220 being bent in either direction.

The extendable protrusions 240 are formed along the tubular portion of the needle 220, proximal to the needle distal end portion 224. The needle distal end portion 224 can be defined as the portion that includes the angled surface 228, generally extending from the heel 232 to the needle tip 230. A length-to-protrusions L_{P} can be defined as the length between the axial position of the heel 232 of the needle distal end portion 124, and the tip 246 of the distal-most protrusion 240a, in the unbent state of the needle 220. When a needle 220 is provided with a single extendable protrusion 240, the length-to-protrusion L_{P} is defined between the heel 232 and the tip 246 of the single protrusion 240. When a needle 220 is provided with a plurality of extendable protrusions 240, as illustrated in FIG. 5A for example, the length-to-protrusions L_{P} is defined between the heel 232 and the tip 246 of the distal-most protrusion 240a.

Various exemplary implementations for tissue perforation systems 200 and/or needles 220 thereof can be referred to, throughout the specification, with superscripts, for ease of explanation of features that refer to such exemplary implementations. It is to be understood, however, that any reference to structural or functional features of any system, device or component, without a superscript, refers to these features being commonly shared by all specific exemplary implementations that can be also indicated by superscripts. In contrast, features emphasized with respect to an exemplary implementation of any system, device or component, referred to with a superscript, may be optionally shared by some but not necessarily all other exemplary implementations. For example, needle 220^{a} is an exemplary implementation of needle 220, and thus can include any of the features described for needle 220 throughout the current disclosure, except that while various materials, dimensions and shapes can be provided for needle wall 221 to allow flexibility thereof, the needle wall 221 of needle 220^{a} can be formed of a material dimensioned to have a needle wall thickness T_{N} small enough to allow it to bend, either passively or actively.

In some examples, the needle 220 is passively bendable, for example when extending through another shaft therearound that assumes a bent shape, or when axially pressed against a surface at a force that facilitates bending thereof. In some examples, the needle 220 is biased towards a bent shape thereof when not bound inside an external constricting surface. In some examples, the needle wall 221 comprises a flexible material, in some examples, the needle wall 221 comprises a shape-memory material, such as Nitinol. In some examples, the needle wall 221 comprises slits arranged in a desired pattern, such as that of known hypo-tubes, to enhance flexibility thereof.

FIG. 6 is a perspective view of an exemplary needle 220^{b}. Needle 220^{b} is an exemplary implementation of needle 220, and thus can include any of the features described for needle 220 throughout the current disclosure, except that the needle 220^{b} comprises further comprises a plurality of successive discrete circumferential slots 280 which are cut (such as by laser-cutting or any other suitable manufacturing procedure) through the needle wall 221, along the side of the needle 220^{b} opposite to the side that includes the extendable protrusions 240.

Each of the slots 280 extends in a transverse direction of the needle 220^{b} between slots ends 282 thereof, spanning more than 180° of the circumference of the needle 220^{b} around the central axis C_{A}. In some examples, slot 280 spans more than 270° around the central axis C_{A}. In some examples, slots 280 extend around the circumference of the needle 220^{b}, for example over at least 200°, at least 220°, at least 280°, at least 300°, at least 220°, or at least 340° circumferentially, leaving an uncut gap between the slot ends 282 that defines the backbone 286. Each circumferential slot 280 can expand, in an unbent state of the needle 220^{b}, between relatively narrow slot ends 282 to a maximal slot width W_{S} at the slot center 284. The extendable protrusions 240 are formed along the backbone 286, such that the slot centers 284 284 are circumferentially opposite to the protrusion tips 246, and wherein the slot width W_{S} is defined in the axial direction (for example, parallel to the central axis C_{A}).

A plurality of circumferential slots 280 can be axially spaced from each other at equal or non-equal distances. The number of circumferential slots 280 can be different than the number of extendable protrusions 240. When a needle 220^{b}is provided with a plurality of extendable protrusions 240, the axial distance between successive protrusions 240 can be different from the axial distance between successive circumferential slots 280.

When the needle 220^{b} is subjected to an axially compressive force, such as a force proximally pushing the needle distal end portion 224 towards a proximal end (not shown) of the needle 220, the edges of the circumferential slots 280 are moved closer together at the slot centers 284. Thus, the slot centers 284 can be narrowed or closed, causing the needle 220^{b} to bend in the direction of the closure.

A protrusion gap width W_{P} can be defined between the cut-out edge 238 and the protrusion free edge 244, in the unbent state of the needle 220. The protrusion gap width W_{P} is measured in a direction that is generally orthogonal to the corresponding region of the cut-out edge 238 and/or the protrusion free edge 244. In some examples, the maximal slot width W_{S} is greater than the protrusion gap width W_{P}. In some examples, the maximal slot width W_{S} is at least two times greater than the protrusion gap width W_{P}. In some examples, the maximal slot width W_{S} is at least three times greater than the protrusion gap width W_{P}. In some examples, the maximal slot width W_{S} is at least five times greater than the protrusion gap width W_{P}.

In some examples, as illustrated in FIG. 6, the extendable protrusions 240 are formed along the heel side 236 such that their protrusion tips 246 are aligned with the heel 232, and the circumferential slots 280 are formed along the tip side 234 such that their slot centers 284 are aligned with the needle tip 230, so as to facilitate a predefined bending direction of the needle 220^{b} in a direction similar to that illustrated in FIG. 5B. In some examples, the extendable protrusions 240 are formed along the tip side 234 such that their slot centers 284 are aligned with the heel 232, and the circumferential slots 280 are formed along the heel side 236 such that their protrusion tips 246 are aligned with the needle tip 230, so as to facilitate a predefined bending direction of the needle 220^{b} in a direction opposite to that illustrated in FIG. 5B.

FIGS. 7A-7L illustrate some steps in a method for utilizing a tissue perforating system 200 for forming an opening within a target tissue. An exemplary implementation of the method is illustrated in FIGS. 7A-7L with respect to forming a leaflet hole inside a host leaflet, which can be performed prior to implanting a guest prosthetic valve inside the host valvular structure. The system 200 can be used to perforate a host leaflet 10, such as a native leaflet 30 or a prosthetic valve leaflet 114 of a previously implanted prosthetic valve. An exemplary system 200^{a}can include a hollow needle 220 extending through an outer shaft 208. The outer shaft 208 can define an outer shaft lumen 210, and the needle 220 can be axially movable through the outer shaft lumen 210, relative to the outer shaft 208. The system 200^{a}can be utilized in a method that includes steps of positioning an expansion member, such as an inflatable balloon 268 (shown in FIG. 7H-7L), inside a puncture formed by the needle 220, for expanding the puncture and forming a wider opening 52 in the host leaflet 10. The expansion member can be optionally advanced through the outer shaft lumen 210, or a different shaft or catheter can be used for delivery the expansion member.

The distal end portion of the system 200 is configured to be inserted into a patient’s vasculature, such as within an ascending aorta, and to be advanced towards the host leaflet 10. Positioning the system 200 relative to the host leaflet 10 may comprise advancing the system 200 toward the leaflet over a guidewire 80. The needle lumen 222 can be configured to accommodate a guidewire 80 that can passed therethrough. In such examples, the guidewire 80 can be inserted into the patient’s vasculature, and then the needle 220 and/or other shafts or tubes of the system 200 may be advanced toward the host leaflet 10 over the guidewire 80.

During delivery, the needle distal end portion 224 can be retained inside the outer shaft lumen 210, such that the sharp needle tip 230 is concealed inside the outer shaft lumen 210, as illustrated in FIG. 7A. This position conceals the needle tip 230 from the surrounding anatomy, to protect the anatomical structures from being engaged or punctured by the needle tip 230 during advancement towards the site of treatment.

The needle 220 can be then axially advanced towards the host leaflet 10, until the needle distal end portion 224 contacts the host leaflet, for example at the needle tip 230 as shown in FIG. 7B. The needle 220 of system 200^{a}may be biased to its unbent state, such that it can retain its straight orientation during advancement thereof, up to the initial contact with the host leaflet 10, maintaining the protrusions 246 substantially flush with the remainder of the needle wall 221, as further shown in FIG. 7B.

The needle 220 can be flexible enough to bend when axially pushed against a surface, such as when the needle 220 is distally pushed against the host leaflet 10. In the state shown in FIG. 7C, the needle 220 is axially pushed against the host leaflet 10 at a force that is high enough to transition the needle 220 to the bent state, yet lower than the force required to penetrate through the tissue of the host leaflet 10. The needle wall 221 can be configured to be flexible enough to bend when an axial force that does not exceed a threshold for penetrating through the host leaflet 10 is applied to the needle 220. This can be achieved, in some examples, by designing the needle wall thickness T_{N} to be thin enough to facilitate such flexibility of the needle. In some examples, the needle wall thickness T_{N} is less than a tenth of the needle outer diameter D_{N}. In some examples, the needle wall thickness T_{N} is not necessarily uniform along the entire length of the needle 220, but may be rather thinned along one or more portions of the needle wall 221 to increase local flexibility at such region(s).

Bending the needle 220, as shown in FIG. 7C, causes the protrusions 240 to extend radially outward, as described above. The axial force applied to the needle 220 can be then increased to a magnitude that allows the needle 220 to puncture the host leaflet 10 to form a pilot puncture 50 within host leaflet 10 as shown in FIG. 7D. In the position of the needle 220 illustrated in FIG. 7D, such as when the host leaflet 10 is positioned around the needle along the section bound between the heel 232 and the distal-most protrusion 240a (that is to say, when the needle 220 does not axially pass a length that is greater than the length-to-protrusion L_{P} after passing the needle distal end portion 224 through the leaflet 10), the size of the pilot puncture 50 can be generally similar to the needle outer diameter D_{N}.

Continued axial translation of the needle 220 through the host leaflet 10, such that at least one extended protrusion 242 is passed therethrough as shown in FIG. 7E, will enlarge the pilot puncture 50 to a size greater than the needle outer diameter D_{N}. For example, the enlarged size of the pilot puncture 50 can be equal to a combination of the needle outer radius R_{N} and the radial distance to the protrusion tip R_{T}, wherein (R_{N} + R_{T}) > 2 * R_{N}. As mentioned above, in some examples, the needle 220 can include a single extendable protrusion 240, which can be sufficient to cut through the leaflet to form a larger pilot puncture 50, relative to the outer diameter D_{N} of the needle. In some examples, as illustrated in FIGS. 7A-7E, the needle can include a plurality of successive extendable protrusions 240, that can be passed through the host leaflet 10 as the needle 220 is further pushed therethrough, cutting the tissue in a saw-like motion.

FIG. 7F shows a portion of the needle 220 passed through the host leaflet 10 after forming the pilot puncture 50, wherein the needle 220, no longer pressed against any surface forcing it to assume a bent state, is free to revert to its biased unbent state. In some examples, advancement of the needle 220 through the host leaflet 10 is performed at a speed fast enough to allow passage of one or more extendable protrusions 240 while still extending radially outwardly to expand the puncture 50, prior to reverting of the needle 220 to the unbent state. As further shown in FIG. 7F, once a portion of the needle 220 is positioned past the host leaflet 10, the guidewire 80 can be advanced through the needle lumen 222 to terminate with guidewire tip 82 at a position distal to the pilot puncture 50 of host leaflet 10.

Subsequent to forming the pilot puncture 50 and optionally advancing the guidewire 80 to extend therethrough, the needle 220 can be optionally retracted, as shown in FIG. 7G, leaving the guidewire 80 extending through the pilot puncture 50.

In some examples, the guidewire 80 can be advanced to terminate distal to the host leaflet 10 after formation of the pilot puncture 50 by the needle 220, as illustrated in FIG. 7F. In some examples, the guidewire 80 can be advanced simultaneously with advancement of the needle 220 towards the host leaflet 10 and/or during formation of the pilot puncture 50.

As mentioned above, the method can further include steps of positioning an expansion member 268 inside the pilot puncture 50. An expansion member can be either part of the system 200, or provided as a separate component advanced into a pilot puncture formed by a needle 220 of the system 200. The expansion member 268 may include and/or be any suitable structure for expanding the pilot puncture 50 to form a leaflet opening 52. In some examples, the expansion member 268 may have a circular profile when in the radially expanded configuration. This is not required of all examples, however, and it additionally is within the scope of the present disclosure that the expansion member 268 may have a non-circular profile when in the radially expanded configuration.

In some examples, the expansion member is an inflatable balloon 268 that can be mounted on a distal portion of a balloon catheter 262. In some examples, a balloon catheter 262 carrying balloon 268 can be advanced over the guidewire 80 towards the pilot puncture 50 formed in host leaflet 10, after retraction of the needle 220, as shown in FIG. 7H. The balloon 268 is configured to transition between a radially deflated state and a radially inflated state. The balloon catheter 262 can define a balloon catheter lumen 266 (indicated, for example, in FIGS. 10A-10B), through which a guidewire 80, and one or more additional shafts of the system 200, can optionally extend. The balloon catheter 262 can extend through a handle 204 of the system 200 (handle 204 indicated, for example, in FIG. 9) and be fluidly connectable to a fluid source (not shown) for inflating the balloon 268. The fluid source comprises an inflation fluid. The term "inflation fluid", as used herein, means a fluid (for example, saline, though other liquids or gas can be used) used for inflating the balloon 268. The inflation fluid source is in fluid communication with the balloon catheter lumen 266, such that fluid from the fluid source can flow through the balloon catheter lumen 266 into balloon 268 to inflate it.

An inflatable balloon 268 of system 200, utilized as a hole-dilating balloon, is different from a typical balloon used for expanding balloon-expandable prosthetic valves or stents, in that while a typical valve-expanding balloon is inflatable to a diameter that can allow expansion of a prosthetic valve to a functional diameter thereof, which can be similar to, or greater than (for example, in the case of valve over-expansion) the diameter of the native annulus in which the valve is deployed, the maximum diameter of a hole-dilating balloon 268 can be significantly smaller, configured to increase the size of a pilot puncture 50 to form a larger leaflet opening 52, optionally without tearing the host leaflet 10 (though in some examples, the host leaflet 10 may be still torn by a balloon 268). In some examples, the maximum diameter to which the hole-dilating balloon 268 can be inflated is equal to or less than 12 mm. In some examples, the maximum diameter to which the hole-dilating balloon 268 can be inflated is equal to or less than 10 mm.

In some examples, a dilator 250 (see FIGS. 7H and 10A-10B) can be positioned distal to the balloon 268 (or other suitable expansion member). The dilator 250 can be either part of the system 200, or provided as a separate component advanced towards a pilot puncture formed by a needle 220 of the system 200. The dilator 250 can be conical or frustoconical in shape, and include a dilator tapering portion 254 terminating at a dilator distal end 252, and a dilator proximal portion 256 that can be coupled to a dilator shaft 258 that extends proximally therefrom. A dilator lumen 260 continuously extends through the dilator shaft 258 and the dilator 250, open ended at the dilator distal end 252. Attachment of the dilator shaft 258 to the dilator proximal portion 256 can be achieved by a variety of methods, such as overmolding, radio-frequency welding, through an adhesive, and/or a combination thereof. In some examples (not illustrated), the dilator shaft 258 can extend through the entire length of the dilator 250, such that a distal end of the dilator shaft 258 is aligned with the dilator distal end 252. In some examples (not illustrated), the dilator shaft 258 is coupled to one or more components, such as collars or other connectors, which are in turn attached to the dilator 250.

In some examples, the balloon 268 is coupled to a distal end portion of the balloon catheter 262 at its proximal end, while the balloon's distal end can be coupled, directly or indirectly, to another component of the system 200, such as the dilator 250 or dilator shaft 258. In the examples illustrated in FIGS. 7H and 10A-10B, the balloon 268 is shown to be coupled to the dilator proximal portion 256. The dilator proximal portion 256 can optionally include an outer step configured to accommodate the distal end of the balloon 268, such that the outer surface of the balloon 268 can be flush or otherwise relatively continuous with the outer surface of the dilator 250.

The outer shaft 208, balloon catheter 262, and/or dilator shaft 258, can be configured to be axially movable relative to each other. For example, a proximally oriented movement of the outer shaft 208 relative to the balloon catheter 262, or a distally oriented movement of the balloon catheter 262 relative to the outer shaft 208, can expose the balloon 268 from the outer shaft 208. Similarly, a proximally oriented movement of the dilator 250 relative to the outer shaft 208, or a distally oriented movement of the outer shaft 208 relative to the dilator 250, can expose the dilator 250 and axially translate it in a desired direction.

In some examples, such as when the balloon 268 is attached at both ends thereof to the dilator 250 and balloon catheter 262, both the dilator 250 with dilator shaft 258 and the balloon catheter 268 can be configured to move simultaneously in the axial direction, without necessarily being axially movable relative to each other, or while axial movement of one relative to the other is limited. In such examples, the system 200 can be designed such that axial movement of the balloon catheter 262 causes the dilator shaft 298 to move therewith, or such that axial movement of one of the dilator shaft 258 or dilator 250 causes the balloon catheter 262 to move therewith.

The proximal ends of various components of system 200, such as outer shaft 208, needle 220, balloon catheter 262, and/or dilator shaft 258, can be coupled to a handle 204 thereof (shown in FIG. 9). During delivery, the handle 204 can be maneuvered by an operator (for example, a clinician or a surgeon) to axially advance or retract components of the system 200, such as outer shaft 208, needle 220, balloon catheter 262, and/or dilator shaft 258, through the patient's vasculature and/or along the target site of treatment, and to expand an expansion member, such as to inflate a balloon 268 mounted on the balloon catheter 262 so as to enlarge a leaflet opening 52, as will be elaborated in further detail below, and to deflate the balloon 268 and optionally retract it.

Subsequent to forming the pilot puncture 50 and extending the guidewire 80 therethrough, and optionally after retraction of the needle 220 as shown in FIG. 7G, a balloon 268 carried over a balloon catheter 262 can be advanced towards the host leaflet 10, as shown in FIG. 7H. In some examples, when a dilator 250 is present distal to the expansion member (such as balloon 268) as also shown in the example illustrated in FIG. 7H, the dilator 250 can be advanced, optionally along with the balloon catheter 262 and balloon 268, towards the host leaflet 10. In such examples, the dilator 250 can be inserted into the pilot puncture 50 to expand the pilot puncture 50, as shown in FIG. 7I. As the dilator 250 is inserted into the host leaflet 10, the inherent resiliency of the leaflet 10 may urge the leaflet 10 radially inwardly against the dilator 250. The dilator 250 can have sufficient stiffness to facilitate advancement thereof through the leaflet 10, wherein the gradually tapering shape of the dilator 250 facilitates expanding the pilot puncture 50 to a greater diameter.

In some examples, the balloon catheter 262 with balloon 268 and/or dilator 250 are advanced towards the pilot puncture 50 of host leaflet 10 over the same guidewire 80 used for advancement of the needle 220 towards the host leaflet 10 for formation of the pilot puncture 50. In some examples, the balloon catheter 262 with balloon 268 and/or dilator 250 are advanced towards the pilot puncture 50 of host leaflet 10 through the lumen 210 of the same outer shaft 208 used for advancement of the needle 220 therethrough towards the host leaflet 10 for formation of the pilot puncture 50.

For example, as illustrated in FIGS. 7G-7H, the needle 220 can be retracted through the outer shaft lumen 210 while the outer shaft 208 remains in position, in the vicinity of the host leaflet 10, with the guidewire 80 extending through the outer shaft lumen 210 into the pilot puncture 50. This allows the balloon catheter 262, and optionally dilator shaft 258, to be advanced towards the pilot puncture 50 of the host leaflet 10 over the guidewire 80, through the lumen 210 of the same outer shaft 208. In some examples, the outer shaft 208 can be retracted along with the needle 220, and then readvanced towards the host leaflet 10 with the balloon catheter 262 and/or dilator shaft 258 extending therethrough.

In a subsequent step of the method, illustrated in FIG. 7J, the balloon 268 may be inserted within the pilot puncture 50, such as by further advancement of the dilator 250 with dilator shaft 258 and/or balloon catheter 262. With the balloon 268 received within the pilot puncture 50, inflating the balloon 268 to transition it from a radially deflated state (FIG. 7J) to a radially inflated state (FIG. 7K) can expand the pilot puncture 50 to form a leaflet opening 52 that is sized to receive the prosthetic valve 100 in the radially compressed or crimped configuration. After the balloon 268 is inflated to form the leaflet opening 52 as shown in FIG. 7K, the balloon 268 is deflated, as shown in FIG. 7L, optionally allowing for insertion of a guest prosthetic valve inside the leaflet opening 52.

In some examples, inflating the balloon 268 within the host leaflet 10 serves to increase a diameter of the pilot puncture 50 such that the resulting leaflet opening 52 is a hole with an increased diameter relative to the pilot puncture 50. In some examples in which the leaflet opening 52 is a hole, the leaflet opening 52 may be a substantially circular hole. In other examples, the leaflet opening 52 may be non-circular (for example, elliptical or asymmetric). In such examples, the diameter of the leaflet opening 52 may refer to any suitable dimension of the leaflet opening 52, such as a minimum diameter of the leaflet opening 52, a maximum diameter of the leaflet opening 52, and/or an average diameter of the leaflet opening 52.

In some examples, inflating the balloon 268 within the host leaflet 10 may cause the host leaflet 10 to rip and/or tear such that the leaflet opening 52 is not a bounded hole. Stated differently, in such examples, the leaflet opening 52 may be formed by a tear that extends from the pilot puncture 50 fully to the free edge of the host leaflet 10 (the coaptation edge of the leaflet).

FIGS. 8A-8F illustrate some steps in a method for utilizing a tissue perforating system 200^{b}for forming an opening within a target tissue. An exemplary implementation of the method is illustrated in FIGS. 8A-8F with respect to forming a leaflet hole inside a host leaflet, which can be performed prior to implanting a guest prosthetic valve inside the host valvular structure. The system 200^{b}can be used to perforate a host leaflet 10, such as a native leaflet 30 or a prosthetic valve leaflet 114 of a previously implanted prosthetic valve.

System 200^{b}can be similar to any example described above with respect to system 200^{a}, except that the needle 220 is biased, in a free state thereof, to the bent state, and the system 200^{b}further comprises a covering member 270 defining a covering member lumen 272, and the needle 220 can be axially movable through the covering member lumen 272, relative to the covering member 270. The covering member 270, which can be in the form of a sleeve or other flexible tubular member, can be axially movable through, and relative to, the outer shaft 208.

The distal end portion of the system 200^{b}is configured to be inserted into a patient’s vasculature, such as within an ascending aorta, and to be advanced towards the host leaflet 10. Positioning the outer shaft 208 and/or the covering member 270 and/or the needle 220, relative to the host leaflet 10, may comprise advancing the outer shaft 208 and/or the covering member 270 and/or the needle 220 toward the leaflet over the guidewire 80 as described above with respect to FIG. 7A for example. As mentioned above, the needle lumen 222 can be configured to accommodate a guidewire 80 that can extend through the needle lumen 222. In such examples, the guidewire 80 can be inserted into the patient’s vasculature, and then the hollow needle 220 and/or other shafts or tubes of the system 200^{b}may be advanced toward the host leaflet 10 over the guidewire 80.

During delivery, the needle distal end portion 224 can be retained inside the covering member lumen 272, retaining the sharp needle tip 230 therein as illustrated in FIG. 8A. This position conceals the needle tip 230 from the surrounding anatomy, to protect the anatomical structures from being engaged or punctured by the needle tip 230 during advancement towards the site of treatment.

During delivery, the covering member 270 can be mostly or entirely retained inside the outer shaft 208. Upon approximation to the host valvular structure 12, at least part of the covering member 270 can be advanced out of the outer shaft 208 towards the host leaflet 10, but without extending all the way to the host leaflet 10. The diameter of the covering member lumen 272 can be substantially similar to the outer diameter D_{N} of the needle 220. The covering member 270 can be flexible enough to passively bend inside the outer shaft 208 while the outer shaft 208 is bent through curved portions of the patient's vasculature, or when the outer shaft 208 is articulated if provided as a steerable outer catheter. Such passive bending of the covering member 270 will cause the flexible needle 220 to passively bend therewith. However, even when the needle 220 is bent inside an outer restricting surface, such as inside the covering member 270, the close proximity of the inner surface of the covering member 270 to the needle wall 221 will prevent the extendable protrusions 240 from protruding radially outward, forcing the protrusions 240 to remain bound inside the covering member lumen 272.

The at least one extendable protrusion 240 of any needle 220 disclosed herein is configured to extend radially outwards, such that the distance R_{P} between the protrusion tip 246 and the central axis C_{A} when the needle 220 is in a bent state, is greater than such distance R_{N} when the needle 220 is in an unbent state, when the needle 220 is not constrained by an outer enclosure at the region of the at least one extendable protrusion 240. In the case of system 200^{b}, the covering member 270 serves as the outer enclosure that prevents the protrusion 240 from extending radially outwards to the distance R_{P} of the protrusion tip 246 from the central axis C_{A}, as long as the covering member 270 is disposed around the needle 220 at the region of protrusion 240, even if the needle 220 is in a bent state.

The needle 220 can be then axially translated in a distal direction towards the leaflet 10, exposing a distal portion thereof out of the covering member lumen 272, as shown in FIG. 8B. As mentioned above, the needle 220 of system 200^{b}is biased, in a free state thereof, to assume a bent state, such as by being formed of a shape memory material (for example, Nitinol) which is shape-set to the bent configuration. Thus, as soon as the needle 220 is exposed out of the covering member 270, the exposed portion, which is no longer bound by the inner surface of the covering member 270, is free to assume its bent shape, allowing the protrusions 240 to extend radially outward as also shown in FIG. 8B.

The needle 220 can be then advanced further to puncture the host leaflet 10 to form a pilot puncture 50 within host leaflet 10 as shown in FIG. 8C. In the position of the needle 220 illustrated in FIG. 8C, such as when the host leaflet 10 is positioned around the needle along the section bound between the heel 232 and the distal-most protrusion 240a (that is to say, when the needle 220 does not axially pass a length that is greater than the length-to-protrusion L_{P} after passing the needle distal end portion 224 through the leaflet 10), the size of the pilot puncture 50 can be generally similar to the needle outer diameter D_{N}.

Continued axial translation of the needle 220 through the host leaflet 10, such that at least one extended protrusion 242 is passed therethrough as shown in FIG. 8D, will enlarge the pilot puncture 50 to a size greater than the needle outer diameter D_{N}. For example, the enlarged size of the pilot puncture 50 can be equal to a combination of the needle outer radius R_{N} and the radial distance to the protrusion tip R_{T}, wherein (R_{N} + R_{T}) > 2 * R_{N}. As mentioned above, in some examples, the needle 220 can include a single extendable protrusion 240, which can be sufficient to cut through the leaflet to form a larger pilot puncture 50, relative to the outer diameter D_{N} of the needle. In some examples, as illustrated in FIGS. 8A-8D, the needle can include a plurality of successive extendable protrusions 240, that can be passed through the host leaflet 10 as the needle 220 is further pushed therethrough, cutting the tissue in a saw-like motion.

FIG. 8E shows an optional subsequent step of distally extending the covering member 270 through the pilot puncture 50 to cover the needle 220, thus folding the extendable protrusions 240 back inwards, and optionally straightening the needle 220 along the distal portion as illustrated. The guidewire 80 can be then advanced through the needle lumen 222 to terminate with guidewire tip 82 at a position distal to the pilot puncture 50 of host leaflet 10.

Subsequent to forming the pilot puncture 50 and optionally advancing the guidewire 80 to extend therethrough, the needle 220 can be optionally retracted along with the covering member 270, as shown in FIG. 8F, leaving the guidewire 80 extending through the pilot puncture 50.

In some examples, the guidewire 80 can be advanced to terminate distal to the host leaflet 10 after formation of the pilot puncture 50 by the needle 220, prior, during, or after optional advancement of covering member 270. In some examples, the guidewire 80 can be advanced simultaneously with advancement of the needle 220 towards the host leaflet 10 and/or during formation of the pilot puncture 50. In some examples, the covering member 270 is not advanced into and through the pilot puncture 50 as shown in FIG. 8E, but the needle 220 can be rather proximally pulled from the host leaflet 10 after formation of the pilot puncture 50, back into the covering member lumen 272.

After retraction of the needle 220 and covering member 270 from the host leaflet 10, an expansion member, such as an inflatable balloon 268 that can be optionally carried by a balloon catheter 262, can be advanced, optionally along with dilator 250 and dilator shaft 258, towards the pilot puncture, and utilized to expand the pilot puncture 50 to form a leaflet opening 52 according to any of the examples described above with respect to FIGS. 7H-7L.

FIG. 9 illustrates an exemplary tissue perforation system 200, which can include the outer shaft 208 and any exemplary needle 220 disclosed herein, extendable through the outer shaft lumen 210 towards the host leaflet 10, for modifying the host leaflet 10. In some examples, a tissue perforation system 200 can include a handle 204, wherein the outer shaft 208 can extend distally from the handle 204. The outer shaft 208 and the needle 220 can be configured to be axially movable relative to each other. For example, a distally oriented movement of the needle 220 relative to the outer shaft 208, can expose the needle distal end portion 224 from the outer shaft lumen 210. The proximal ends of the outer shaft 208 and the needle 220 can be coupled to the handle 204. During delivery through the patient's vasculature, the handle 204 can be maneuvered by an operator (for example, a clinician or a surgeon) to axially advance or retract components of the tissue perforation system 200, such as the outer shaft 208 or any other component passing therethrough, including needle 220.

The handle 204 can include a steering mechanism configured to adjust the curvature of the distal end portion of the tissue perforation system 200. In the illustrated example, the handle 204 can include an adjustment member, such as the illustrated rotatable knob 206a, which in turn is operatively coupled to the proximal end portion of a pull wire. The pull wire can extend distally from the handle 204 through the outer shaft 208 and has a distal end portion affixed to the outer shaft 208 at or near the distal end of the outer shaft 208. Rotating the knob 206a can increase or decrease the tension in the pull wire, thereby adjusting the curvature of the distal end portion of the stabilized tissue perforation system 200. Further details on steering or flex mechanisms for the delivery apparatus can be found in U.S. Patent No. 9,339,384, which is incorporated by reference herein.

The handle 204 can further include needle advancement mechanism which can be optionally operable by a knob of the handle, such as the illustrated rotatable knob 206b. A proximal end of the needle 220 can be operatively connected to a knob 206b to effect axial movement of the needle 220.

The handle can include additional adjustment mechanisms controllable by additional knobs to maneuver additional components of the tissue perforation system 200, such as axial movement of a covering member 270 when present, and/or axial movement of a dilator shaft 258 and/or balloon catheter 262 when present, relative to other shafts of the system 200. The terms "tissue perforation system 200" and "system 200", as used herein, are interchangeable.

FIGS. 10A and 10B are cross-sectional views of a distal portion of an exemplary tissue perforation system 200^{c}, shown in an unbent state and a bent state, respectively, of a needle 220 thereof. System 200^{c}is an exemplary implementation of system 200, and thus can include any of the features described for system 200 throughout the current disclosure, except that the system 200^{c}further includes an expansion member 268, such as inflatable balloon 268 mounted on balloon catheter 262, extending through the outer shaft lumen 210, between the needle 220 and the outer shaft 208. The system 200^{c}can optionally comprise the dilator 250 attached to dilator shaft 258, and the balloon 268 can be optionally disposed between a distal end of the balloon catheter 262 and the dilator proximal portion 256 according to any of the examples described above for dilator 250, dilator shaft 258, and/or balloon 268. The hollow needle 220 includes one or more extendable protrusions 240, and can be implemented according to any of the examples described above.

The needle 220 extends through the dilator lumen 260 as illustrated in FIGS. 10A-10B, and is configured to be axially movable in the distal and proximal direction relative to any of the dilator shaft 258 and/or the balloon catheter 262. The dilator shaft 278 can extend through the balloon catheter lumen 266, and may be sized such that an annular space is formed within balloon catheter lumen 266 between an inner surface of the balloon catheter 262 and an outer surface of the dilator shaft 258 along the length of balloon catheter 262. This annular space is in fluid communication with one or more balloon catheter inflation openings 264 exposed to an internal cavity of the balloon 268, which can be in fluid communication with a fluid source (for example, a syringe or a pump) that can inject inflation fluid (for example, saline) into the balloon 268, so as to inflate the balloon 268, for example during formation of leaflet opening 52. The pressure of the inflation fluid within balloon 268 may provide the force that allows it to dilate a leaflet opening 52. Further, the balloon catheter lumen 266 may be configured to withdraw fluid from the balloon 268 through the balloon catheter inflation opening(s) 264, to deflate the balloon 268.

In the illustrated example, the balloon 268 is shown to be coupled to a distal end portion of the balloon catheter 262 at its proximal end, and to the dilator proximal portion 256 at the balloon's distal end. The dilator proximal portion 256 can optionally include an outer step configured to accommodate the distal end of the balloon 268, such that the outer surface of the balloon 268 can be flush or otherwise relatively continuous with the outer surface of the dilator 250.

FIGS. 11A-11G illustrate some steps in a method for utilizing a system 200^{c}for forming an opening within a target tissue. An exemplary implementation of the method is illustrated in FIGS. 11A-11G with respect to forming a leaflet hole inside a host leaflet 10, which can be performed prior to implanting a guest prosthetic valve inside the host valvular structure. The system 200^{c} can be used to perforate a host leaflet 10, such as a native leaflet 30 or a prosthetic valve leaflet 114 of a previously implanted prosthetic valve.

The distal end portion of the system 200^{c}is configured to be inserted into a patient’s vasculature, such as within an ascending aorta, and to be advanced towards the host leaflet 10. Positioning the outer shaft 208 and/or the dilator 250 and/or the needle 220, relative to the host leaflet 10, may comprise advancing the outer shaft 208 and/or the dilator shaft 258 and/or the needle 220 toward the leaflet over the guidewire 80 as described above with respect to FIGS. 7A or 8A for example. As mentioned above, the needle lumen 222 can be configured to accommodate a guidewire 80 that can extend through the needle lumen 222. In such examples, the guidewire 80 can be inserted into the patient’s vasculature, and then the hollow needle 220 along with the dilator 250, dilator shaft 258, balloon catheter 262, and/or outer shaft 208, may be advanced toward the host leaflet 10 over the guidewire 80.

During delivery, the needle distal end portion 224 can be retained inside the dilator lumen 260, retaining the sharp needle tip 230 therein as illustrated in FIG. 11A. This position conceals the needle tip 230 from the surrounding anatomy, to protect the anatomical structures from being engaged or punctured by the needle tip 230 during advancement towards the site of treatment.

The needle 220 of system 200^{c}is biased, in a free state thereof, to the bent state, in a similar manner to that described above with respect to the needle 220 of system 200^{b}. The diameter of the dilator lumen 260 can be substantially similar to the outer diameter D_{N} of the needle 220. The dilator shaft 258 can be flexible enough to passively bend inside the outer shaft 208 and/or balloon catheter 262 while the outer shaft 208 and/or balloon catheter 262 are bent through curved portions of the patient's vasculature, or when the outer shaft 208 is articulated if provided as a steerable outer catheter. Such passive bending of the dilator shaft 258 will cause the flexible needle 220 to passively bend therewith. However, as described above with respect to a needle 220 used in combination with a covering member 270, even when the needle 220 is bent inside an outer restricting surface, such as inside the dilator shaft 258 and/or inside dilator 250, the close proximity of the inner surface of the dilator shaft 258 and/or inside dilator 250 to the needle wall 221 will prevent the extendable protrusions 240 from protruding radially outward, forcing the protrusions 240 to remain bound inside the dilator lumen 260.

The needle 220 can be then axially translated in a distal direction towards the leaflet 10, exposing a distal portion thereof out of the dilator lumen 260, as shown in FIG. 11B. As mentioned above, the needle 220 of system 200^{c}is biased, in a free state thereof, to assume a bent state, such as by being formed of a shape memory material (for example, Nitinol) which is shape-set to the bent configuration. Thus, as soon as the needle 220 is exposed out of the dilator 250, the exposed portion, which is no longer bound by the inner surface of the dilator 250 and/or dilator shaft 258, is free to assume its bent shape, allowing the protrusions 240 to extend radially outward as also shown in FIG. 11B.

Thus. in the case of system 200^{c}, the dilator 250 and/or dilator shaft 258 serves as the outer enclosure that prevents the at least one protrusion 240 of the needle 220 from extending radially outwards to the distance R_{P} of the protrusion tip 246 from the central axis C_{A}, as long as the dilator 250 and/or dilator shaft 258 is disposed around the needle 220 at the region of protrusion 240, even if the needle 220 is in a bent state.

The needle 220 can be then advance further to puncture the host leaflet 10 to form a pilot puncture 50 within host leaflet 10 as shown in FIG. 11C. In the position of the needle 220 illustrated in FIG. 11C, such as when the host leaflet 10 is positioned around the needle along the section bound between the heel 232 and the distal-most protrusion 240a (that is to say, when the needle 220 does not axially pass a length that is greater than the length-to-protrusion L_{P} after passing the needle distal end portion 224 through the leaflet 10), the size of the pilot puncture 50 can be generally similar to the needle outer diameter D_{N}.

Continued axial translation of the needle 220 through the host leaflet 10, such that at least one extended protrusion 242 is passed therethrough as shown in FIG. 11D, will enlarge the pilot puncture 50 to a size greater than the needle outer diameter D_{N}. For example, the enlarged size of the pilot puncture 50 can be equal to a combination of the needle outer radius R_{N} and the radial distance to the protrusion tip R_{T}, wherein (R_{N} + R_{T}) > 2 * R_{N}. As mentioned above, in some examples, the needle 220 can include a single extendable protrusion 240, which can be sufficient to cut through the leaflet to form a larger pilot puncture 50, relative to the outer diameter D_{N} of the needle. In some examples, as illustrated in FIGS. 11A-11D, the needle can include a plurality of successive extendable protrusions 240, that can be passed through the host leaflet 10 as the needle 220 is further pushed therethrough, cutting the tissue in a saw-like motion.

FIG. 11E shows a subsequent step of distally extending the dilator 250 through the pilot puncture 50 to cover the needle 220, thus folding the extendable protrusions 240 back inwards, and optionally straightening the needle 220 along the distal portion as illustrated. As the dilator is passed through the pilot puncture 50, it can also further expand the pilot puncture 50 to a greater diameter. Advancement of the dilator 250 with dilator shaft 258 and/or balloon catheter 262 can continue until the balloon 268 is inserted within the pilot puncture 50. With the balloon 268 received within the pilot puncture 50, inflating the balloon 268 to transition it from a radially deflated state (FIG. 11E) to a radially inflated state (FIG. 11F) can expand the pilot puncture 50 to form a leaflet opening 52 that is sized to receive the prosthetic valve 100 in the radially compressed or crimped configuration. After the balloon 268 is inflated to form the leaflet opening 52 as shown in FIG. 11F, the balloon 268 is deflated, as shown in FIG. 11G, optionally allowing for insertion of a guest prosthetic valve inside the leaflet opening 52.

In some examples, a system 200^{c}further includes a covering member 270 extending through the dilator lumen 260 and around the needle 220. The covering member 270 of system 200^{c}can be implemented according to any of the examples described for covering member 270 of system 200^{b}, and can be axially movable relative to the dilator 250, such that the covering member 270 can define the outer enclosure around the needle 220.

Any exemplary system 200 disclosed herein may be configured to form the leaflet opening 52 in any of a variety of host valvular structures 12. In the examples of FIGS. 7A-7L, 8A-8F, or 11A-11G, the host valvular structure 12 can be the valvular structure 113 of a previously implanted prosthetic valve, such as the prosthetic valve 100a of FIG. 3. In such examples, using the a system 200 as described herein to form the leaflet opening 52 in a previously implanted prosthetic valve may be followed by steps for implanting a guest prosthetic valve 100b within the previously implanted prosthetic valve 100a (for example, via a ViV procedure).

Similarly, the host valvular structure 12 in the examples of FIGS. 7A-7L, 8A-8F, or 11A-11G, can be a valvular structure 29 of a native heart valve, such as the native aortic valve 20 shown in FIGS. 2A-2B. In such examples, the system 200 can be configured to puncture a native leaflet 30 of the native aortic valve 20. In some examples, the host valvular structure and/or the native valve may refer to another valve of a patient’s heart, such as a mitral valve, a pulmonary valve, or a tricuspid valve.

While illustrated and described above with respect to forming a leaflet opening 52 within a host leaflet 10, it is to be understood that any exemplary system 200 disclosed herein may be configured to form a tissue opening through other tissues in a patient's body. For example, prosthetic devices can be delivered to the left atrium or the left ventricle in a transseptal approach, wherein a system 200 is passed through the vena cava, into the right atrium, and through the interatrial septum tissue. Such delivery approaches require puncturing the interatrial septum. Thus, in some examples, a system 200 may be utilized to form an opening through the interatrial septum, for example at the site of the fossa ovalis, which is a region of the septum containing tissue of lesser thickness than is typical of the rest of the septum. Thus, any example of a system 200 described herein can be utilized in a manner similar to that described with respect to FIGS. 7A-7L, 8A-8F, or 11A-11G, to form a tissue opening, equivalent to leaflet opening 52 described with respect to FIGS. 7A-7L, 8A-8F, or 11A-11G, in a target tissue, equivalent to a host leaflet 10 described with respect to FIGS. 7A-7L, 8A-8F, or 11A-11G.

In some examples, some or all of the components of any exemplary system 200 described herein can be part of a delivery assembly that includes a delivery apparatus carrying a prosthetic valve (examples not shown explicitly). Similarly, a tissue perforation system 200 according to any example of the current disclosure, can be used for implantation of other prosthetic devices aside from prosthetic valves, such as stents or grafts.

A tissue perforation system 200 can be part of a delivery apparatus utilized, for example, to deliver a prosthetic aortic valve for mounting against the native aortic annulus or against a prosthetic valve previously implanted in a native aortic valve, to deliver a prosthetic mitral valve for mounting against the native mitral annulus or against a prosthetic valve previously implanted in a native mitral valve, or to deliver a prosthetic valve for mounting against any other native annulus or against a prosthetic valve previously implanted in any other native valve.

In some examples, after forming the leaflet opening 52, and optionally after deflating the balloon 268, a guest prosthetic valve 100 can be positioned in the valvular structure 12 in a compressed state thereof, and expanded therein to implant the guest prosthetic valve 100 inside the host valvular structure. In some examples, the guest prosthetic valve 100 can be positioned inside a leaflet opening 52 in a radially compressed state thereof, and expanded therein in a manner that modifies the host leaflet 10. Radially expanding the guest prosthetic valve 100 can be performed in any suitable manner, such as using any suitable valve expansion technique and/or mechanism that is known to the art. In some examples, radial expansion of the guest prosthetic valve 100 can be achieved by inflating an inflatable valve-expanding balloon on which the guest prosthetic valve is mounted. As mentioned above, in contrast to the hole-dilating balloon 268, the valve-expanding balloon (not shown) is configured to expand to a diameter which is significantly greater than a maximum diameter of the hole-dilating balloon 268.

In some examples, a tissue perforation system 200 is part of a delivery assembly that further includes the guest prosthetic valve 100 carried, in a radially compressed state thereof, over a component of the system 200. Exemplary delivery assemblies that include perforating members that can be implemented in the form of a needle, a first balloon that can be a hole-dilating balloon 268, and a second balloon that can be valve-expanding balloon, are described in U.S. Provisional Application Nos. 63/447,453 and 63/447,457, each of which is incorporated herein by reference in its entirety.

In some examples, a tissue perforation system 200 can be retracted from the host valvular structure 12 and the patient's body, optionally subsequent to deflation of balloon 268, while the guidewire 80 remains in position, extending through the leaflet opening 52. Positioning a guest prosthetic valve within the leaflet opening can be performed, in such examples, by advancing the guest prosthetic valve into the leaflet opening via over the same guidewire 80.

In some examples, more than one guidewire can be utilized in a method that includes forming the leaflet opening 52 by a tissue perforation system 200 and positioning a guest prosthetic valve 100 therein. For example, a first guidewire 80 can be utilized in a method of forming a leaflet opening 52 by the stabilized tissue perforation system 200 following the steps described with respect to FIGS. 7A-7L, 8A-8F, or 11A-11G herein, after which the system 200 can be retracted along with guidewire 80, and a separate guidewire can be then used for advancing a guest prosthetic valve in the host valvular structure. In some examples, a separate guidewire over which a guest prosthetic valve can be advanced, can extend alongside the guidewire 80 over which the system 200 extends.

In some examples, the guest prosthetic valve can be a mechanically-expandable prosthetic valve and radial expansion thereof can be achieved by actuating a mechanical actuator of the guest prosthetic valve to mechanically expand a frame of the guest prosthetic valve. In some examples, the guest prosthetic valve can be a self-expandable prosthetic valve that can be retained during delivery toward the host valvular structure in a capsule or other restraint disposed therearound, and valve expansion can be achieved by removing the capsule or other restraint from the guest prosthetic valve to allow it to radially self-expand within the host valvular structure.

With the guest prosthetic valve received within the leaflet opening 52, radial expansion thereof can serve to increase a size of the leaflet opening and/or to tear the leaflet. As a result, the valve's radial expansion can serve to modify the host leaflet 10 such that the leaflet does not obstruct a cell opening in a frame of the guest prosthetic valve or at least increases the exposed area of the host valvular structure and the guest prosthetic valve that is not covered or obstructed by the modified host leaflet 10 to permit access and sufficient perfusion to the adjacent coronary artery.

While methods disclosed herein can refer to forming a leaflet opening 52 in a host leaflet 10, prior to positioning and expanding a prosthetic valve 100, it is to be understood that any of the methods can comprise, in some examples, repeating one or more steps disclosed throughout the current specification to form a plurality of openings in the host valvular structure. For example, steps described above with respect to FIGS. 7A-7L, 8A-8F, or 11A-11G, can be performed for forming a first leaflet opening in a first host leaflet, after which the system 200 can be retracted from the first host leaflet and steered toward another host leaflet, after which the same steps can be repeated to form a second leaflet opening within the second host leaflet. The procedure can be optionally repeated to form further leaflet openings, such as a third leaflet opening in a third host leaflet.

In some examples, forming more than one leaflet opening, such as forming the second leaflet opening, can provide further access and/or fluid paths through the frame of the guest prosthetic valve. For example, radially expanding the guest prosthetic valve 100 within the first leaflet opening may push the second host leaflet against the frame of the guest prosthetic valve such that the second leaflet opening is aligned with cell opening(s) of the frame of the guest prosthetic valve. Thus, the second leaflet opening can provide additional unobstructed paths through the frame of the guest prosthetic valve. Moreover, in an example in which the host valve is a previously implanted prosthetic valve, expanding the guest prosthetic valve within the first leaflet opening can trap the second leaflet opening between the respective frames of the host prosthetic valve and the guest prosthetic valve, thereby providing additional access and/or flow paths through each of the frames.

Thus, forming the second leaflet opening can ensure that a greater number of cell openings of the frame are uncovered, and/or that a greater proportion of the frame is uncovered, relative to an example in which only one leaflet is punctured to form a leaflet opening. This may be particularly beneficial in examples in which the frame of a host prosthetic valve extends axially in a downstream direction beyond one or both of the coronary arteries when the guest prosthetic valve is implanted within a native heart valve.

For example, in some patient anatomies, the left coronary artery is positioned lower (that is, proximate to the host valvular structure) than the right coronary artery. In such examples, the right coronary artery may be sufficiently far from the host valvular structure that implanting the guest prosthetic heart valve within the host valvular structure does not limit access and/or perfusion to the right coronary artery. Accordingly, forming a single leaflet opening in the host valvular structure may be sufficient to ensure access and/or perfusion to both coronary arteries, provided that the leaflet opening is formed and/or positioned to ensure access to the left coronary artery.

In other examples, however, each of the left and right coronary arteries may be positioned sufficiently proximate to the host valvular structure that forming a single leaflet opening in the host valvular structure is insufficient to ensure access to both coronary arteries. In such examples, forming two leaflet openings in respective leaflets of the previously implanted prosthetic heart valve may ensure the ability for future access into both coronary arteries or perfusion through the frame to both coronary arteries during the diastole phase of the cardiac cycle. For example, the host valvular structure can be modified such that the guest prosthetic valve is implanted by being expanded in a leaflet opening of a first host leaflet that faces the left coronary artery, and such that the second leaflet opening is formed in a second host leaflet that faces the right coronary artery (or vice-versa).

In some examples, forming the first leaflet opening can be performed prior to forming the second leaflet opening. In other examples, forming the second leaflet opening can be performed prior to forming the first leaflet opening. In some examples, the order of forming leaflet openings is chosen such that the final leaflet opening is formed in the host leaflet in which a guest prosthetic valve 100 is to be positioned and expanded.

It is to be understood that the guest prosthetic valve 100 is not limited to being implanted within an opening 52 of a leaflet. For example, in cases where the system 200 is utilized to form a full tear in a host leaflet that extends to the coaptation edge of the leaflet, the guest prosthetic valve 100 can be positioned at a location between the leaflets of the host valvular structure 12 and then expanded. In such cases, the opening 52 may provide sufficient open space through which blood may flow into the coronary ostia, and/or through which additional access devices, such as coronary catheters, can pass during future interventional procedures.

As mentioned, any system and method of the current specification can be utilized for forming a leaflet opening 52 in a host leaflet 10 which can be either a native leaflet 30 or a prosthetic valve leaflet 114 of a previously implanted prosthetic valve, such as prosthetic valve 100a of FIG. 3, such as in the case of ViV procedures. FIG. 12 shows a previously implanted prosthetic valve 100a subsequent to forming the leaflet opening 52, for example subsequent to the method described above with respect to FIGS. 7A-7L, 8A-8F, or 11A-11G. FIG. 13 shows a configuration in which a second prosthetic valve 100b has been expanded within the leaflet opening 52 of a host prosthetic valve 100a. In the example of FIG. 13, the guest prosthetic valve 100b is the same type of valve as the host prosthetic valve 100a. It is to be understood, however, that ViV procedures may be similarly applied to any other suitable valvular structures, such as different prosthetic valves and/or native heart valves. For example, the guest prosthetic valve 100b need not be the same type of valve as the host prosthetic valve 100a.

In the example of FIG. 12, when the prosthetic valve leaflets 114a of the previously implanted prosthetic valve 100a are pressed against the frame 102a, the leaflet opening 52 provides a partial access into the frame 102a, but the leaflet opening 52 may not be sufficiently large to completely uncover any of the cell openings 112a of the frame 102a.

As shown in FIG. 13, however, fully expanding the guest prosthetic valve 100b within the leaflet opening 52 further expands and/or tears the leaflet opening 52 such that several cell openings 112a of the frame 102a of the host prosthetic valve 100a and several cell openings 112b of the frame 102b of the guest prosthetic valve 100b are fully uncovered by the leaflets 114a. In some examples, this may result from the frame 102b of the guest prosthetic valve 100b pushing the leaflet 114a comprising the leaflet opening 52 downwardly (toward the inflow ends of the prosthetic valves 100a, 100b) such that one or more cell openings 112a are unobstructed by the leaflet 114a. In some examples, expanding the frame 102b within the leaflet 114a comprising the leaflet opening 52 may rip and/or tear this leaflet 114a such that the leaflet 114a cannot obstruct one or more cell openings 112a.

In some examples, the guidewire 80 of any system 200 or method described herein, can be used as a perforating or lacerating member for forming a pilot puncture 50 prior to and/or simultaneously with the needle 220. In such examples, the guidewire 80 can be a relatively stiff wire having a distal tip 82 configured to pierce the host leaflet 10 when the guidewire 80 is pressed against the leaflet. In some examples, the guidewire 80 can include a radio-frequency (RF) energy delivery tip 82 to assist with penetration through the leaflet tissue. For this purpose, a suitable RF energy device may be coupled to the guidewire 80, and the RF energy device can apply the RF energy to the guidewire tip 82 to penetrate the host leaflet 10.

In any examples disclosed herein wherein a guidewire is used to puncture a leaflet, the guidewire can be coupled to a source of RF energy that applies RF energy to the tip of the guidewire. In some examples, the guidewire 80 is used as a perforating member that can be used in addition needle 220, such that the guidewire 80 can form an initial puncture via a sharp tip 82 or an RF energy delivery tip 82, followed by penetration of needle 220 into the leaflet 10 to form the pilot puncture 50, or a pilot puncture 50 which is greater in size than an initial puncture formed by the guidewire tip 82.

In some examples, the guidewire tip 82 is not necessarily sharp enough or otherwise configured to puncture through the host leaflet 10, in which case the guidewire 80 can be utilized for advancement of the system 200 and/or needle 220 and other shafts thereof toward the valvular structure 12, but terminate in proximity of the host leaflet 10 without piercing through it, and the needle 220 can be then advanced into the leaflet 10 to form the pilot puncture 50.

While a hole-dilating balloon is described above and illustrated for expanding a pilot puncture 50 to form a leaflet opening 52, it is to be understood that other types of expansion member 268 can be used instead of a balloon in any of the methods and/or systems described herein. For example, U.S. Provisional Application Nos. 63/335,739, which is incorporated herein by reference in its entirety, describes an expandable frame that can be used as an expansion member 268 instead of a valve-expanding balloon.

Any of the tools, devices, apparatuses, etc. herein can be sterilized (for example, with heat, radiation, and/or chemicals, etc.) to ensure they are safe for use with patients, and any of the methods herein can include sterilization of the associated assembly, device, apparatus, etc. as one of the steps of the method. Examples of radiation for use in sterilization include, without limitation, gamma radiation and ultra-violet radiation. Examples of chemicals for use in sterilization include, without limitation, ethylene oxide and hydrogen peroxide.

### Some Examples of the Disclosed Implementations

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. A tissue perforating system comprising:

an outer shaft;

a needle axially movable through and relative to the outer shaft, the needle comprising:

a needle distal end portion comprising a needle tip;

a needle wall defining a needle lumen around a central axis of the needle; and

at least one extendable protrusion extending continuously from a protrusion base at the needle wall, along at least one protrusion free edge, to a protrusion tip;

wherein the needle is configured to transition between an unbent state and a bent state; and

wherein when the needle is in the bent state, the protrusion tip radially protrudes from the needle wall.

Example 2. The system of any example herein, particularly of example 1, wherein, when the needle is in the bent state, the protrusion tip is separated from the needle wall by a first radial distance, and when the needle is in the bent state, the protrusion tip is separated from the needle wall by a second radial distance, wherein the second radial distance is greater than the first radial distance.

Example 3. The system of any example herein, particularly of example 1, wherein, when the needle is in the bent state, the protrusion tip is separated from the central axis of the needle by a first radial distance, and when the needle is in the bent state, the protrusion tip is separated from the central axis of the needle by a second radial distance, wherein the second radial distance is greater than the first radial distance.

Example 4. The system of any example herein, particularly of any one of examples 1 to 3, wherein, when the needle is in the bent state, the at least one extendable protrusion extends at an angle with respect to the needle wall.

Example 5. The system of any example herein, particularly of any one of examples 1 to 4, wherein the needle is configured to move from the unbent state to the bent state when a portion of the needle is uncovered by the outer shaft.

Example 6. The system of any example herein, particularly of any one of examples 1 to 5, wherein the at least one extendable protrusion comprises a plurality of extendable protrusions.

Example 7. The system of any example herein, particularly of example 6, wherein the plurality of extendable protrusions are axially spaced from each other.

Example 8. The system of any example herein, particularly of any one of examples 1 to 7, wherein the protrusion tip is distal to the protrusion base.

Example 9. The system of any example herein, particularly of any one of examples 1 to 8, wherein the at least one extendable protrusion is V-shaped.

Example 10. The system of any example herein, particularly of any one of examples 1 to 9, wherein the radial distance between the protrusion tip and the central axis in the unbent state of the needle is greater than an outer radius of the needle.

Example 11. The system of any example herein, particularly of example 10, wherein the radial distance between the protrusion tip and the central axis in the bent state of the needle is equal to the outer radius of the needle.

Example 12. The system of any example herein, particularly of any one of examples 1 to 11, wherein the at least one extendable protrusion is integrally formed with the needle wall.

Example 13. The system of any example herein, particularly of any one of examples 1 to 12, wherein the at least one extendable protrusion is laser cut from the needle wall along the at least one protrusion free edge.

Example 14. The system of any example herein, particularly of any one of examples 1 to 13, wherein the needle tip is a sharp tip configured to penetrate through a target tissue.

Example 15. The system of any example herein, particularly of example 14, wherein the target tissue is a host leaflet of a host valvular structure.

Example 16. The system of any example herein, particularly of example 15, wherein the host valvular structure is a native valvular structure of native heart valve.

Example 17. The system of any example herein, particularly of example 15, wherein the host valvular structure is a valvular structure of previously implanted prosthetic valve that is implanted within a native heart valve.

Example 18. The system of any example herein, particularly of examples 16 or 17, wherein the native heart valve is an aortic valve.

Example 19. The system of any example herein, particularly of any one of examples 1 to 18, wherein the needle distal end portion comprises an angled surface extending between the needle tip and a heel radially opposite to the needle tip.

Example 20. The system of any example herein, particularly of example 19, wherein the protrusion tip of at least one of the at least one extendable protrusion is aligned with the needle tip in the unbent state of the needle.

Example 21. The system of any example herein, particularly of examples 19 or 20, wherein the protrusion tip of at least one of the at least one extendable protrusion is aligned with the heel in the unbent state of the needle.

Example 22. The system of any example herein, particularly of any one of examples 1 to 21, wherein the needle further comprises a plurality of successive discrete circumferential slots extending through the needle wall.

Example 23. The system of any example herein, particularly of example 22, wherein at least one of the plurality of circumferential slots extends in a transverse direction of the needle, orthogonal to the central axis.

Example 24. The system of any example herein, particularly of examples 22 or 23, wherein at least one of the plurality of circumferential slots spans more than 180° of a circumference of the needle.

Example 25. The system of any example herein, particularly of any one of examples 22 to 24, wherein at least one of the plurality of circumferential slots has a maximal slot width at a slot center thereof.

Example 26. The system of any example herein, particularly of example 25, wherein the slot center is radially opposite to the protrusion tip.

Example 27. The system of any example herein, particularly of examples 25 or 26, wherein the needle wall further comprises at least one cut-out edge that is parallel to the protrusion free edge of a corresponding one of the at least one extendable protrusion, and wherein the maximal slot width is greater than a protrusion gap width defined between the cut-out edge and the corresponding protrusion free edge.

Example 28. The system of any example herein, particularly of example 27, wherein the maximal slot width is at least two times greater than the protrusion gap width.

Example 29. The system of any example herein, particularly of example 27, wherein the maximal slot width is at least three times greater than the protrusion gap width.

Example 30. The system of any example herein, particularly of example 27, wherein the maximal slot width is at least five times greater than the protrusion gap width.

Example 31. The system of any example herein, particularly of any one of examples 1 to 30, further comprising a covering member defining a covering member lumen through which the needle extends.

Example 32. The system of any example herein, particularly of example 31, wherein the needle is axially movable through and relative to the covering member.

Example 33. The system of any example herein, particularly of example 31 or 32, wherein the covering member is axially movable through and relative to the outer shaft.

Example 34. The system of any example herein, particularly of any one of examples 31 to 33, wherein the covering member defines an outer enclosure configured to allow the needle tip to radially protrude from the needle wall when the at least one protrusion is not constrained by the outer enclosure.

Example 35. The system of any example herein, particularly of any one of examples 1 to 34, further comprising an expansion member configured to expand a pilot puncture formed in a target tissue by the needle.

Example 36. The system of any example herein, particularly of example 35, wherein the needle is configured to be axially advanced over a guidewire, and wherein the expansion member is configured to be axially advanced over the same guidewire.

Example 37. The system of any example herein, particularly of examples 35 or 36, wherein the expansion member is axially movable through, and relative to, the outer shaft.

Example 38. The system of any example herein, particularly of any one of examples 35 to 37, further comprising a balloon catheter defining a balloon catheter lumen, wherein the expansion member comprises a balloon mounted on the balloon catheter and in fluid communication with the balloon catheter lumen, the balloon configured to transition between deflated and inflated states thereof.

Example 39. The system of any example herein, particularly of example 38, wherein the balloon catheter is extendable through the outer shaft.

Example 40. The system of any example herein, particularly of examples 38 or 39, wherein the needle is axially movable relative to the balloon catheter.

Example 41. The system of any example herein, particularly of any one of examples 38 to 40, further comprising a dilator attached to a dilator shaft extending proximally therefrom through the balloon catheter lumen, wherein the needle extends through a dilator lumen defined by the dilator and the dilator shaft.

Example 42. The system of any example herein, particularly of example 41, wherein the needle is axially movable through and relative to the dilator.

Example 43. The system of any example herein, particularly of examples 41 or 42, wherein the needle is axially movable through and relative to the dilator shaft.

Example 44. The system of any example herein, particularly of any one of examples 41 to 43, wherein at least part of an outer enclosure is defined by the dilator , configured to allow the needle tip to radially protrude from the needle wall when the at least one protrusion is not constrained by the outer enclosure.

Example 45. The system of any example herein, particularly of any one of examples 41 to 43, wherein at least part of an outer enclosure in defined by the dilator shaft, configured to allow the needle tip to radially protrude from the needle wall when the at least one protrusion is not constrained by the outer enclosure.

Example 46. The system of any example herein, particularly of any one of examples 41 to 45, wherein the dilator comprises a dilator tapering portion.

Example 47. The system of any example herein, particularly of example 46, wherein the dilator further comprises a dilator proximal portion which is proximal to the dilator tapering portion.

Example 48. The system of any example herein, particularly of any one of examples 41 to 47, wherein the balloon is attached on one end to the balloon catheter, and on an opposite end to the dilator.

Example 49. The system of any example herein, particularly of any one of examples 41 to 47, wherein the balloon is attached on one end to the balloon catheter, and on an opposite end to the dilator shaft.

Example 50. The system of any example herein, particularly of any one of examples 1 to 49, wherein the needle is biased to the bent state in a free state thereof.

Example 51. The system of any example herein, particularly of any one of examples 1 to 50, wherein the needle is made of a shape-memory material.

Example 52. The system of any example herein, particularly of example 51, wherein the shape-memory material comprises Nitinol.

Example 53. A method comprising:

advancing a tissue perforating system comprising a needle, over a guidewire, to a host valvular structure;

transitioning the needle to a bent state thereof, such that at least one extendable protrusion of the needle extends radially outwards relative to a central axis defined by the needle;

forming, with a needle tip of the needle, a pilot puncture within a host leaflet of the host valvular structure; and

distally advancing the needle to cut through the host leaflet with the at least one extendable protrusion, thereby enlarging the pilot puncture.

Example 54. The method of any example herein, particularly of example 53, wherein the enlarging the pilot puncture comprises enlarging the pilot puncture to a size that is greater than an outer diameter of the needle.

Example 55. The method of any example herein, particularly of examples 53 or 54, wherein the transitioning the needle to the bent state comprises transitioning the needle to an uncovered bent state.

Example 56. The method of any example herein, particularly of any one of examples 53 to 55, wherein the at least one extendable protrusion extends continuously from a protrusion base at a needle wall of the needle, to a protrusion tip, defining at least one protrusion free edge between the protrusion base and the protrusion tip.

Example 57. The method of any example herein, particularly of example 56, wherein the protrusion tip is distal to the protrusion base.

Example 58. The method of any example herein, particularly of examples 56 or 57, wherein the needle comprises a needle distal end portion defining an angled surface that extends from the needle tip to a heel radially opposite to the needle tip.

Example 59. The method of any example herein, particularly of example 58, wherein a radius of curvature of a heel side of the needle that is aligned with the heel, is greater than a radius of curvature of a tip side of the needle that is aligned with the needle tip, in the bent state of the needle.

Example 60. The method of any example herein, particularly of example 59, wherein the protrusion tip of the at least one extendable protrusion is radially aligned with the heel.

Example 61. The method of any example herein, particularly of example 58, wherein a radius of curvature of a tip side of the needle that is aligned with the needle tip, is greater than a radius of curvature of a heel side of the needle that is aligned with the heel, in the bent state of the needle.

Example 62. The method of any example herein, particularly of example 61, wherein the protrusion tip of the at least one extendable protrusion is radially aligned with the needle tip.

Example 63. The method of any example herein, particularly of any one of examples 57 to 62, wherein the transitioning the needle to the bent state comprises distancing the protrusion tip to a distance that is greater than an outer radius of the needle, relative to the central axis.

Example 64. The method of any example herein, particularly of any one of examples 53 to 63, wherein the needle comprises a needle lumen sized to allow passage of the guidewire therethrough.

Example 65. The method of any example herein, particularly of any one of examples 53 to 64, wherein the at least one extendable protrusion is V-shaped.

Example 66. The method of any example herein, particularly of any one of examples 53 to 65, wherein the at least one extendable protrusion is integrally formed with the needle wall.

Example 67. The method of any example herein, particularly of any one of examples 53 to 66, wherein the at least one extendable protrusion is formed at a side of the needle that is opposite to the direction of bending of the needle during the transitioning of the needle to the bent state.

Example 68. The method of any example herein, particularly of any one of examples 53 to 67, wherein the at least one extendable protrusion comprises a plurality of extendable protrusions, and wherein the distally advancing the needle to cut through the host leaflet with the at least one extendable protrusion comprises distally advancing the needle to cut through the host leaflet with the at least some of the plurality of extendable protrusions.

Example 69. The method of any example herein, particularly of example 68, wherein the plurality of extendable protrusions are axially spaced from each other.

Example 70. The method of any example herein, particularly of any one of examples 53 to 67, wherein the tissue perforating system further comprises an outer shaft defining an outer shaft lumen, and wherein the needle is disposed inside the outer shaft lumen during the advancing of the tissue perforating system to the host valvular structure.

Example 71. The method of any example herein, particularly of example 70, wherein the advancing the tissue perforating system to the host valvular structure comprises retaining the needle tip inside the outer shaft lumen.

Example 72. The method of any example herein, particularly of examples 70 or 71, wherein the transitioning the needle to the bent state comprises distally advancing the needle so as to expose the needle tip out of the outer shaft lumen.

Example 73. The method of any example herein, particularly of any one of examples 53 to 72, wherein the transitioning the needle to the bent state comprises pressing the needle tip against the host leaflet at a force that facilitates bending of the needle, yet is lower than a force required to penetrate the host leaflet by the needle tip.

Example 74. The method of any example herein, particularly of example 73, wherein the forming the pilot puncture comprises applying a distally oriented force on the needle, sufficient to facilitate penetration of the needle tip through the host leaflet.

Example 75. The method of any example herein, particularly of any one of examples 53 to 72, wherein the needle is configured to bias towards the bent state in a free state of the needle.

Example 76. The method of any example herein, particularly of example 75, wherein the needle is made of a shape-memory material.

Example 77. The method of any example herein, particularly of example 76, wherein the shape-memory material comprises Nitinol.

Example 78. The method of any example herein, particularly of any one of examples 75 to 77, wherein the advancing the tissue perforating system to the host valvular structure comprises retaining the at least one extendable protrusion inside an outer enclosure of the tissue perforating system.

Example 79. The method of any example herein, particularly of example 78, wherein the needle is axially movable relative to the outer enclosure.

Example 80. The method of any example herein, particularly of examples 78 or 79, wherein the transitioning the needle to the bent state comprises exposing a portion of the needle that comprises the at least one extendable protrusion out of the outer enclosure, thereby allowing the exposed portion of the needle to assume the bent state.

Example 81. The method of any example herein, particularly of any one of examples 78 to 80, wherein the tissue perforating system further comprises a covering member defining a covering member lumen through which the needle extends.

Example 82. The method of any example herein, particularly of example 81, wherein the covering member defines the outer enclosure.

Example 83. The method of any example herein, particularly of any one of examples 73 to 82, further comprising passing the guidewire through the pilot puncture to terminate distally to the pilot puncture of the host leaflet.

Example 84. The method of any example herein, particularly of example 83, wherein the passing the guide through the pilot puncture comprises passing the guidewire through the needle.

Example 85. The method of any example herein, particularly of example 83 or 84, further comprising, after the passing the guidewire through the pilot puncture, retrieving the needle while maintaining the guidewire extending through the pilot puncture.

Example 86. The method of any example herein, particularly of example 85, further comprising, subsequent to the retrieving the needle, advancing an expansion member, over the guidewire, towards the host leaflet.

Example 87. The method of any example herein, particularly of example 86, further comprising, subsequent to the advancing the expansion member, positioning the expansion member inside the pilot puncture, in a compacted state of the expansion member.

Example 88. The method of any example herein, particularly of example 87, further comprising, subsequent to the positioning the expansion member inside the pilot puncture, expanding the expansion member to expand the pilot puncture and form a leaflet opening within the host leaflet.

Example 89. The method of any example herein, particularly of example 88, wherein the expansion member comprises a balloon mounted on a balloon catheter, wherein the compacted state of the expansion member is a deflated state of the balloon, and wherein the expanding the expansion member comprises inflating the balloon.

Example 90. The method of any example herein, particularly of example 89, further comprising, subsequent to the forming the pilot puncture and prior to the positioning the balloon inside the pilot puncture, passing a dilator through the pilot puncture, thereby further expanding the pilot puncture.

Example 91. The method of any example herein, particularly of any one of examples 78 to 80, wherein the tissue perforating system further comprises an expansion member, and wherein the needle is axially movable relative to the expansion member.

Example 92. The method of any example herein, particularly of example 91, further comprising, after the advancing the needle to cut through the host leaflet with the at least one extendable protrusion, positioning the expansion member inside the pilot puncture, in a compacted state of the expansion member.

Example 93. The method of any example herein, particularly of example 92, further comprising, subsequent to the positioning the expansion member inside the pilot puncture, expanding the expansion member to expand the pilot puncture and form a leaflet opening within the host leaflet.

Example 94. The method of any example herein, particularly of example 93, wherein the expansion member comprises a balloon mounted on a balloon catheter, wherein the compacted state of the expansion member is a deflated state of the balloon, and wherein the expanding the expansion member comprises inflating the balloon.

Example 95. The method of any example herein, particularly of example 94, wherein the tissue perforating system further comprises a dilator defining a dilator lumen, and wherein the needle extends through the dilator lumen.

Example 96. The method of any example herein, particularly of example 95, further comprising, subsequent to the advancing the needle to cut through the host leaflet with the at least one extendable protrusion and prior to the positioning the balloon inside the pilot puncture, passing the dilator through the pilot puncture, thereby further expanding the pilot puncture.

Example 97. The method of any example herein, particularly of examples 95 or 96, wherein at least part of the outer enclosure in defined by the dilator.

Example 98. The method of any example herein, particularly of any one of examples 90 or 94-97, wherein the dilator is attached to a dilator shaft extending proximally therefrom, through a lumen of the balloon catheter.

Example 99. The method of any example herein, particularly of any one of examples 90 or 94-98, wherein the dilator comprises a dilator tapering portion terminating at a dilator distal end.

Example 100. The method of any example herein, particularly of any one of examples 90 or 94-99, wherein the balloon is attached at a proximal end thereof to the balloon catheter, and at a distal end of the balloon to the dilator.

Example 101. The method of any example herein, particularly of any one of examples 88-90 or 93-97, further comprising, subsequent to the expanding the expansion member, transitioning the expansion member back to its compacted state.

Example 102. The method of any example herein, particularly of any one of examples 88-90 or 93-97 or 101, further comprising, subsequent to the forming the leaflet opening, positioning a guest prosthetic valve in a radially compressed state thereof within the host valvular structure, and radially expanding the guest prosthetic valve.

Example 103. The method of any example herein, particularly of example 102, wherein the positioning the guest prosthetic valve within the host valvular structure comprises positioning the guest prosthetic valve within the leaflet opening.

Example 104. The method of any example herein, particularly of example 102, wherein the positioning the guest prosthetic valve within the host valvular structure comprises positioning the guest prosthetic valve between host leaflets of the host valvular structure.

Example 105. The method of any example herein, particularly of any one of examples 102 to 104, wherein the radially expanding the guest prosthetic valve comprises inflating a valve-expanding balloon over which the guest prosthetic valve is disposed.

Example 106. The method of any example herein, particularly of any one of examples 102 to 104, wherein the radially expanding the guest prosthetic valve comprises actuating a mechanical actuator of the guest prosthetic valve.

Example 107. The method of any example herein, particularly of any one of examples 102 to 104, wherein the guest prosthetic valve is a self-expandable prosthetic valve, and wherein radially expanding the guest prosthetic valve comprises removing a restraint from around the guest prosthetic valve.

Example 108. The method of any example herein, particularly of any one of examples 53 to 107, wherein the host valvular structure is a native valvular structure of native heart valve.

Example 109. The method of any example herein, particularly of any one of examples 53 to 107, wherein the host valvular structure is a valvular structure of previously implanted prosthetic valve that is implanted within a native heart valve.

Example 110. The method of any example herein, particularly of examples 108 or 109, wherein the native heart valve is an aortic valve.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A tissue perforating system comprising:
an outer shaft;
a needle axially movable through and relative to the outer shaft, the needle comprising:
a needle distal end portion comprising a needle tip;
a needle wall defining a needle lumen around a central axis of the needle; and
at least one extendable protrusion extending continuously from a protrusion base at the needle wall, along at least one protrusion free edge, to a protrusion tip;
wherein the needle is configured to transition between an unbent state and a bent state; and
wherein, when the needle is in the bent state, the protrusion tip radially protrudes from the needle wall.

2. The system of claim 1, wherein, when the needle is in the bent state, the protrusion tip is separated from the needle wall by a first radial distance, and when the needle is in the bent state, the protrusion tip is separated from the needle wall by a second radial distance, wherein the second radial distance is greater than the first radial distance.

3. The system of claim 1, wherein, when the needle is in the bent state, the protrusion tip is separated from the central axis of the needle by a first radial distance, and when the needle is in the bent state, the protrusion tip is separated from the central axis of the needle by a second radial distance, wherein the second radial distance is greater than the first radial distance.

4. The system of claim 1, wherein, when the needle is in the bent state, the at least one extendable protrusion extends at an angle with respect to the needle wall.

5. The system of claim 1, wherein the needle distal end portion comprises an angled surface extending between the needle tip and a heel radially opposite to the needle tip.

6. The system of claim 5, wherein the protrusion tip of at least one of the at least one extendable protrusion is aligned with the heel in the unbent state of the needle.

7. The system of claim 1, further comprising a covering member defining a covering member lumen through which the needle extends.

8. The system of claim 7, wherein the covering member defines an outer enclosure configured to allow the needle tip to radially protrude from the needle wall when the at least one protrusion is not constrained by the outer enclosure.

9. The system of claim 1, further comprising a balloon catheter defining a balloon catheter lumen, and a balloon mounted on the balloon catheter and in fluid communication with the balloon catheter lumen, the balloon configured to transition between deflated and inflated states thereof.

10. The system of claim 9, further comprising a dilator attached to a dilator shaft extending proximally therefrom through the balloon catheter lumen, wherein the needle extends through a dilator lumen defined by the dilator and the dilator shaft.

11. The system of claim 10, wherein at least part of an outer enclosure is defined by the dilator, configured to allow the needle tip to radially protrude from the needle wall when the at least one protrusion is not constrained by the outer enclosure.

12. The system of claim 10, wherein at least part of an outer enclosure is defined by the dilator shaft, configured to allow the needle tip to radially protrude from the needle wall when the at least one protrusion is not constrained by the outer enclosure.

13. The system of claim 1, wherein the needle is biased to the bent state in a free state thereof.

14. A method comprising:
advancing a tissue perforating system comprising a needle, over a guidewire, to a host valvular structure;
transitioning the needle to a bent state thereof, such that at least one extendable protrusion of the needle extends radially outwards relative to a central axis defined by the needle;
forming, with a needle tip of the needle, a pilot puncture within a host leaflet of the host valvular structure; and
distally advancing the needle to cut through the host leaflet with the at least one extendable protrusion, thereby enlarging the pilot puncture.

15. The method of claim 14, wherein the transitioning the needle to the bent state comprises uncovering the needle.

16. The method of claim 14, wherein the at least one extendable protrusion extends continuously from a protrusion base at a needle wall of the needle, to a protrusion tip, defining at least one protrusion free edge between the protrusion base and the protrusion tip, wherein the protrusion tip is distal to the protrusion base, and wherein the transitioning the needle to the bent state comprises distancing the protrusion tip to a distance that is greater than an outer radius of the needle, relative to the central axis.

17. The method of claim 14, wherein the at least one extendable protrusion comprises a plurality of extendable protrusions, and wherein the distally advancing the needle to cut through the host leaflet with the at least one extendable protrusion comprises distally advancing the needle to cut through the host leaflet with the at least some of the plurality of extendable protrusions.

18. The method of claim 14, wherein the transitioning the needle to the bent state comprises pressing the needle tip against the host leaflet at a force that facilitates bending of the needle, yet is lower than a force required to penetrate the host leaflet by the needle tip.

19. The method of claim 18, wherein the forming the pilot puncture comprises applying a distally oriented force on the needle, sufficient to facilitate penetration of the needle tip through the host leaflet.

20. The method of claim 14, wherein the needle is configured to bias towards the bent state in a free state of the needle.

21. The method of claim 20, wherein the advancing the tissue perforating system to the host valvular structure comprises retaining the at least one extendable protrusion inside an outer enclosure of the tissue perforating system.

22. The method of claim 21, wherein the transitioning the needle to the bent state comprises exposing a portion of the needle that comprises the at least one extendable protrusion out of the outer enclosure, thereby allowing the exposed portion of the needle to assume the bent state.
